(19) United States
(12) Patent Application Publication
(10) Pub. No.: US 2023/0220466 A1
(43) Pub. Date: Jul. 13, 2023

(54) IMMUNE CELL SEQUENCING METHODS

(71) Applicant: The Regents of the University of California, Oakland, CA (US)

(72) Inventor: Christopher Vollmers, Santa Cruz, CA (US)

(21) Appl. No.: 17/641,935

(22) PCT Filed: Sep. 10, 2020
(86) PCT No.: PCT/US2020/050184

§ 371 Date: Mar. 10, 2022

Related U.S. Application Data
(60) Provisional application No. 62/898,420, filed on Sep. 10, 2019.

(51) Int. Cl. C12Q 1/6881

(52) U.S. Cl. CPC C12Q 1/6881; C12Q 1/6844

(57) ABSTRACT

Provided are immune cell RNA sequencing methods. In some embodiments, the methods comprise producing a circularized DNA comprising a complementary DNA (cDNA) and a known heterologous sequence, wherein the cDNA is produced from an immune cell RNA. Such methods further comprise performing rolling circle amplification using the circularized DNA as template to produce a concatemer comprising repeating segments comprising the cDNA and the known heterologous sequence. Such methods further comprise sequencing the concatemer or fragments thereof. Also provided are methods comprising producing immune cell RNA sequencing reads using a R2C2 sequencing method, extracting HLA reads from the sequencing reads, and producing allele-specific HLA sequences from the extracted HLA reads. Also provided are computer-readable media, systems, compositions and kits that find use, e.g., in practicing the methods of the present disclosure.

### CROSS-REFERENCE TO RELATED APPLICATIONS

This application claims the benefit of U.S. Provisional Patent Application No. 62/898,420, filed Sep. 10, 2019, which application is incorporated herein by reference in its entirety.

### INTRODUCTION

The human immune system relies on highly diverse and complex receptors to protect us from a wide array of pathogens. The transcripts encoding these immune receptors are of great interest to basic and translational research as well as diagnostic and other clinical purposes. However, RNA-seq, the current gold-standard for whole transcriptome analysis, falls short of describing these immune receptors completely and accurately. Accurate and deep full-length cDNA sequencing of immune cell transcriptomes could overcome this shortfall by providing 1) the isoforms of surface receptors targeted in immunotherapy, 2) allele-resolved HLA transcript sequences central to self/non-self discrimination, and 3) B cell receptor (BCR) and T cell receptor (TCR) repertoires instrumental to the adaptive immune response to pathogens and diseases such as cancer.

First, full-length cDNA sequencing should be capable of investigating the transcript isoforms of surface receptors expressed by B cells which have important roles in the immune response but are also themselves targets in the treatment of B cell derived leukemia. For example, current antibody and Chimeric Antigen Receptor (CAR) T-cell therapies against B-cell acute lymphoblastic leukemia (B-ALL) target epitopes of CD19, CD20, and CD22. However, evidence is accumulating that these epitopes might be absent in some isoforms expressed by these genes. Detailed analysis of this absence and possible consequences is hampered by the inability of RNA-seq to resolve transcript isoforms. Determining isoform-level transcriptomes of healthy and cancerous immune cells might therefore inform treatment decisions and future development.

Second, full-length cDNA sequencing could be used to accurately determine the sequence and identity of alleles of HLA genes. HLA genes encode for the Major Histocompatibility Complex (MHC) group of immune receptors which are instrumental in the presentation of antigens on the cell surface. Most cells express HLA class I transcripts (A, B, and C) which encode for proteins that present fragments of other endogenous proteins and can therefore alert the adaptive immune system of viral infections hijacking the cellular machinery. Professional antigen presenting cells like B cells, dendritic cells, and Macrophages also express HLA-class II (including DPA1, DPB1, DRA, DRB1, DQA1, and DQB1) transcripts which encode proteins that present foreign antigens on the cell surface and can lead to the activation of the adaptive immune system. Determining the identities of HLA alleles that are present within an individual’s genome (HLA-typing) is of central importance to establish compatibility of donor and recipient for organ and bone-marrow transplantation because the genes encoding HLA transcripts are highly diverse throughout the human population and will therefore be recognized as foreign if not properly matched.

Currently, HLA-typing is performed in clinical laboratories by amplifying the genomic DNA encoding these genes and sequencing the resulting amplicons with short read sequencers. This targeted approach is required because although all HLA genes are expressed highly by immune cells in the blood and are therefore easily captured by RNA-seq, even sophisticated computational tools relying on complex workflows and statistically rigorous frameworks struggle to process RNA-seq data and provide reliable allelic identities. Further, these tools rely on databases of known HLA allele sequences which prevents them for identifying so far unknown HLA alleles. Extracting accurate and full-length HLA allele sequences from full-length cDNA sequences could therefore dramatically simplify the computational task of determining the identities of HLA alleles present in a sample, independent of whether these HLA alleles have previously been identified.

Third, accurate full-length cDNA sequencing should also be capable of determining adaptive immune receptor sequences, including BCR heavy and light chains and TCR alpha and beta transcripts. Due to their complexity, these transcripts likely pose the biggest challenge in transcriptome analysis. These receptor transcripts contain a constant and a variable region. The constant region determines the type and characteristics of the receptor and the variable region determines its binding affinity. The exon encoding these variable regions is generated through the process of somatic VDJ recombination which randomly recombines of a pool of similar but distinct V, D, and J gene segments. Each developing T or B cell uses this somatic recombination process unique to these cell types to rearrange non-functional loci into two functional genes (B cells: heavy/light (kappa or lambda); T cells: alpha/beta). Each T and B cell thereby generates a unique TCR and BCR. The repertoires of these transcripts present in blood or tissue samples carry a large amount of information on the composition of the loci from which they are expressed, as well as the activation state, clonal composition (including malignant clones in Leukemia), and basic biological processes of the adaptive immune system.

Currently, targeted Adaptive Immune Receptor Repertoire sequencing assays (AIRR-seq) methods are routinely employed to sequence these transcripts and investigate the human immune system. Specialized assays are required for this task because the diversity and unique nature of these transcripts make them practically impossible to analyze at full length using standard RNA-seq protocols. Further, the majority of these assays are based on primers against known V segments and therefore potentially biased against so far unknown V segments. The ability to instead extract these full-length transcripts in an unbiased way from whole transcriptome full-length cDNA sequencing would greatly simplify workflows and expand the information that can be recovered from non-targeted transcriptome analysis.

### SUMMARY

Provided are immune cell RNA sequencing methods. In some embodiments, the methods comprise producing a circularized DNA comprising a complementary DNA (cDNA) and a known heterologous sequence, wherein the cDNA is produced from an immune cell RNA. Such methods further comprise performing rolling circle amplification using the circularized DNA as template to produce a concatemer comprising repeating segments comprising the cDNA and the known heterologous sequence. Such methods further comprise sequencing the concatemer or fragments thereof. Also provided are methods comprising producing immune cell RNA sequencing reads using a R2C2 sequencing method, extracting HLA reads from the sequencing reads, and producing allele-specific HLA sequences from the extracted HLA reads. Also provided are computer-readable media, systems, compositions and kits that find use, e.g., in practicing the methods of the present disclosure.

### BRIEF DESCRIPTION OF THE FIGURES

FIG. 1 Analysis of the adaptive immune system through high-throughput sequencing. Schematic of experimental design. DNA and RNA extracted from a PBMC sample underwent several library preparation protocols to generate AIRR-seq, Smart-seq2, Smar2C2, and R2C2 libraries that were sequenced on Illumina and ONT sequencers.

FIG. 2 Isoform level transcriptome analysis. Swarmplots of the A) accuracy [Matchesl(Matches+Mismatches+Indels)] and B) transcript length [Matches+Mismatches] of R2C2 reads (with and without UMI) and isoforms. Red line indicates median. C) Normalized read coverage around newly identified splice sites, Transcription Start Sites (TSSs), and polyA sites determined by either all (splice sites), pTSS (Transcription start sites) or pPolyA (PolyA sites) Smar2C2 reads. D) The CD19 gene locus is shown in a genome-browser view. Gencode annotation (top), Mandalorion isoforms (center) and R2C2 reads (bottom) are shown. Direction of shown features is indicated by color (“top strand” => blue, “bottom strand” => yellow).

FIG. 3 Allele-resolved isoform expression and sequences. A) Computational strategy for determining allele-resolved isoforms. B-E) SNHG5, HLA-A, HLA-DPA1, and HLA-DRA gene loci are shown in a genome-browser view. Gencode annotation (top), Mandalorion isoforms (center) and allele-resolved R2C2 reads (bottom) are shown. Direction of shown features is indicated by color (“top strand” => blue, “bottom strand” => yellow). Mismatches of R2C2 reads to the genome reference are shown in black. Both HLA-A and HLA-DPA show allele-resolved differential expression of isoforms with alternative polyA sites. E) Arrows indicate allele-specific variants in the HLA-DRA gene.

FIG. 4 R2C2 repertoires have advantages and disadvantages compared to AIRR-seq repertoires: A) CDR3 lengths, B) Isotype distribution, and C) V segment usage are shown for BCR IGH repertoires are determined by AIRR-seq (top) and R2C2 (bottom). In C), different bar colors indicate different V segment alleles with grey indicating ambiguous allele calls. Red arrows indicate V segments where AIRR-seq fails to identify an allele unambiguously but R2C2 succeeds. Black errors indicate rarely recombined V segments that AIRR-seq but not R2C2 detected.

FIG. 5 Somatic Hypermutation can be characterized within R2C2-based IGH repertoires. A) Mismatch mutations per IGH sequence as determined by IgBlast are shown as swarmplots separated by Isotype (IGHM, IGHA1), isoform (membrane-bound, secreted), and technology (R2C2, AIRR-seq) and compared to TCR-beta sequences. Averages are indicated by horizontal lines. B) The pattern of mutation locations in V segments in AIRR-seq, TMI-seq and R2C2 sequences is shown for 1000 randomly sampled IGHA1 or TCR-beta sequences.

FIG. 6 Clonal lineages can be measured by R2C2-based repertoires. A) Lineages shared between AIRR-seq and R2C2-based repertoires are indicated by connections within this Circos plot. Abundance of each lineage within each repertoire is shown as a histogram on the outside of the circle. Color of connecting lines and histogram bars indicates the isotype of a lineage (Red: IGHA, Black: IGHM, Blue: IGHG, Green: IGHD) B) The distribution of the percentage of mutations in an IGH sequence being shared with IGH sequences within the same or different lineages is shown as swarm-plots for R2C2-based and AIRR-seq repertoires. C) The size of lineages ordered by rank is shown for the R2C2-based repertoire (red) and 100 repertoires subsampled from the AIRR-seq repertoire to match the R2C2-based repertoires depth (grey).

### DETAILED DESCRIPTION

Before the methods, computer-readable media, systems, compositions and kits of the present disclosure are described in greater detail, it is to be understood that the methods, computer-readable media, systems, compositions and kits are not limited to particular embodiments described, as such may, of course, vary. It is also to be understood that the terminology used herein is for the purpose of describing particular embodiments only, and is not intended to be limiting, since the scope of the methods, computer-readable media, systems, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, computer-readable media, systems, compositions and kits. The upper and lower limits of these smaller ranges may independently be included in the smaller ranges and are also encompassed within the methods, computer-readable media, systems, compositions and kits, subject to any specifically excluded limit in the stated range. Where the stated range includes one or both of the limits, ranges excluding either or both of those included limits are also included in the methods, computer-readable media, systems, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, computer-readable media, systems, compositions and kits belong. Although any methods, compositions and kits similar or equivalent to those described herein can also be used in the practice or testing of the methods, computer-readable media, systems, compositions and kits, representative illustrative methods, computer-readable media, systems, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, computer-readable media, systems, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, computer-readable media, systems, compositions and kits, which are, for clarity, described in the context of separate embodiments, may also be provided in combination in a single embodiment. Conversely, various features of the methods, computer-readable media, systems, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, computer-readable media, systems, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods

The present disclosure provides immune cell ribonucleic acid (RNA) sequencing methods. In certain aspects, such methods comprise producing a circularized deoxyribonucleic acid (DNA) comprising a complementary DNA (cDNA) and a known heterologous sequence, wherein the cDNA is produced from an immune cell RNA. Such methods further comprise performing rolling circle amplification using the circularized DNA as template to produce a concatemer comprising repeating segments comprising the cDNA and the known heterologous sequence. Such methods further comprise sequencing the concatemer or fragments thereof. In other aspects, provided are methods that comprise producing immune cell RNA sequencing reads using a Rolling Circle Amplification to Concatemeric Consensus (R2C2) sequencing method, extracting HLA reads from the sequencing reads, and producing allele-specific HLA sequences from the extracted HLA reads.

The methods of the present disclosure find use in a variety of contexts. For example, the methods enable identification of allele-specific expression of RNA isoforms by immune cells (e.g., isoforms of receptors targeted by a therapy, e.g., CD19 isoforms, and/or any other receptor of interest). Identifying allele-specific expression of isoforms represents a formidable challenge for RNA-seq data alone making long reads indispensable for this type of analysis. In addition, the methods enable the generation of accurate allele-resolved full-length isoforms including the isoforms of all major HLA genes, in turn enabling accurate high-resolution HLA-typing. In contrast to short-read RNA-seq based HLA-typing which requires a reference database, these HLA allele sequences can be also used to discover thus far unknown HLA alleles. Moreover, the methods enable identification of tens of thousands of full-length adaptive immune receptor (AIR) transcripts which may be compiled into repertoires containing valuable data regarding the state of the adaptive immune system. Such methods represent a convenient alternative to specialized AIRR-seq assays for the generation of AIRR-data which has been used to detect minimal residual disease, organ rejection, etc., or for basic research to track B cell clonal lineages or analyze immune aging or class-switching. The ability of the present methods to investigate BCR IGH repertoires as demonstrated herein shows that they can replace specialized AIRR-seq for many purposes. It can be used to determine V segment compositions, isotype usage, and somatic hypermutation levels and patterns, as well as the clonal structure of a population of B cells. In contrast to AIRR-seq methods, generation of R2C2-based repertoires requires no specific primer sets which makes it a powerful tool for the investigation of not only human but vertebrate adaptive immune receptor diversity. To summarize, the present methods provide for the in-depth analysis of the human immune system and may replace or enhance specialized RNA-seq, HLA-typing and AIRR-seq approaches in the analysis of clinical samples. Details regarding the methods of the present disclosure will now be described.

In certain aspects, the methods of the present disclosure comprise producing a circularized DNA comprising a complementary DNA (cDNA) and a known heterologous sequence. In the context of messenger RNA, the cDNA includes at least the complement of a coding portion (e.g., the entire coding portion) of the mRNA from which the cDNA is produced/derived. The cDNA may be the initial cDNA molecule produced upon reverse transcription of the mRNA, or the cDNA may be a copy of such an initial molecule, e.g., an amplicon of such an initial molecule.

cDNAs may be produced by performing a reverse transcription reaction on an RNA sample of interest, where the RNA sample of interest comprises RNAs from one or more types of immune cells. The one or more types of immune cells may comprise T cells, B cells, natural killer (NK) cells, macrophages, monocytes, neutrophils, dendritic cells, mast cells, basophils, eosinophils, and any combination thereof. In certain embodiments, the RNA sample of interest comprises RNAs isolated from whole blood samples, a fraction of whole blood comprising peripheral blood mononuclear cells (e.g., blood plasma), serum, a peripheral blood mononuclear cell (PBMC) sample, urine, buffy coat, synovial fluid, bone marrow, cerebrospinal fluid, saliva, lymph fluid, seminal fluid, vaginal secretions, urethral secretions, exudate, transdermal exudates, pharyngeal exudates, nasal secretions, sputum, sweat, bronchoalveolar lavage, tracheal aspirations, fluid from joints, or vitreous fluid. Immune cells, e.g., T cells, can also be obtained from biological samples which may be derived from, for example, solid tissue samples. T-cells may be helper T cells (effector T cells or Th cells), cytotoxic T cells (CTLs), memory T cells, and regulatory T cells. In some embodiments, peripheral blood mononuclear cells (PBMCs) are isolated by techniques known to those of skill in the art, e.g., by Ficoll-Hypaque® density gradient separation.

The immune cell RNAs of interest may be isolated from a single immune cell or a plurality of immune cells, e.g., PBMCs, cultured immune cells, immune cells from a tissue, an organ, or the like. In certain aspects, the immune cell RNA sample of interest is isolated from a cell(s), tissue, organ, and/or the like of an animal. In some embodiments, the animal is a mammal (e.g., a mammal from the genus Homo, a rodent (e.g., a mouse or rat), a dog, a cat, a horse, a cow, or any other mammal of interest). In some embodiments, the mammal is a human.

Approaches, reagents and kits for isolating RNA from immune cell samples of interest are known in the art and commercially available. For example, kits for isolating RNA from immune cell samples of interest include the RNeasy®, QIAamp®, QIAprep® and QIAquick® nucleic acid isolation/purification kits by Qiagen, Inc. (Germantown, Md); the ChargeSwitch®, Purelink®, GeneCatcher® nucleic acid isolation/purification kits by Life Technologies, Inc. (Carlsbad, CA); the NucleoMag®, NucleoSpin®, and NucleoBond® nucleic acid isolation/purification kits by Clontech Laboratories, Inc. (Mountain View, CA), and TRlzol™ reagent by Invitrogen™. In certain aspects, the RNA is isolated from a fixed biological sample, e.g., formalin-fixed, paraffin-embedded (FFPE) tissue. Genomic DNA from FFPE tissue may be isolated using commercially available kits - such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

Reverse transcription may be accomplished using a suitable primer (e.g., an oligo dT or other suitable primer) and a suitable polymerase (e.g., reverse transcriptase) to carry out a first-strand cDNA synthesis reaction. Reagents and kits for carrying out such reverse transcription are readily available and include, e.g., the SMART® cDNA Library Construction Kit or the In-Fusion® SMARTer® Directional cDNA Library Construction Kit available from Clontech Laboratories, Inc. (Mountain View, CA); and the SuperScript IV First-Strand Synthesis System available from ThermoFisher Scientific.

In some embodiments, reverse transcription is carried out using an oligo(dT) primer having a 5′ non-hybridizing region that includes a sequence heterologous to the RNA. Such embodiments may further include a polymerase extension reaction to produce the cDNA, where upon reaching the 5′ end of the RNA template, a terminal transferase activity of the polymerase (e.g., MMLV reverse transcriptase) adds a few additional nucleotides (e.g., deoxycytidine) to the 3′ end of the newly synthesized cDNA strand. These bases may then function as a template switch (TS) oligonucleotide-anchoring site. Upon base pairing between the TS oligo and the appended additional nucleotides (e.g., deoxycytidine stretch), the reverse transcriptase “switches” template strands, from the template RNA to the TS oligo, and continues replication to the 5′ end of the TS oligo. By doing so, the resulting cDNA contains the complete 5′ end of the transcript, and one or more heterologous sequences (e.g., index sequence, or the like) may be added to one or both ends of the reverse transcription product. Along with tagging of the cDNA 3′ end by oligo dT primers, this approach makes it possible to efficiently amplify the entire full-length transcript pool in a completely sequence-independent manner.

As used herein, a “heterologous” sequence is a nucleic acid sequence not found within or immediately adjacent to the RNA or cDNA in nature. By “known” heterologous sequence is meant the practitioner of the subject methods knows that the heterologous sequence is being added to the RNA and/or cDNA (e.g., during reverse transcription and/or cDNA amplification) prior to or during circularization. The known heterologous sequence may be a sequence such as an index sequence. In certain aspects, the known heterologous sequence comprises a sequencing adapter or portion thereof. By “sequencing adapter” is meant a nucleic acid domain selected from: a domain (e.g., a “capture site” or “capture sequence”) that specifically binds to a surface-attached sequencing platform oligonucleotide; a sequencing enzyme-binding domain (e.g., a nucleic acid domain that binds an enzyme (e.g., a helicase) employed in a nanopore-based sequencing system; a sequencing primer binding domain; a barcode domain (e.g., a domain that uniquely identifies the sample source of the nucleic acid being sequenced to enable sample multiplexing by marking every molecule from a given sample with a specific barcode or “tag”); a barcode sequencing primer binding domain (a domain to which a primer used for sequencing a barcode binds); an index sequence (e.g., a molecular index tag, such as a randomized tag of 4, 6, or other number of nucleotides); a complement of any such domains; or any combination thereof. In some embodiments, the known heterologous sequence is a combination of heterologous sequences.

In certain aspects, the methods include PCR amplifying the cDNA (e.g., full-length cDNA) prior to circularization. In some embodiments, amplification of the cDNA is facilitated by the presence of at least one known heterologous sequence present at one or both ends of the cDNA. For example, the amplification may be carried out using a PCR primer pair in which one primer hybridizes to a known heterologous sequence (e.g., an index sequence) at one end of the cDNA and the other primer hybridizes to a known heterologous sequence (e.g., an index sequence) at the opposite end of the cDNA. The resulting amplicons will include the cDNA and a known heterologous sequence or portion thereof at each end.

Circularizing a nucleic acid that includes the cDNA and a known heterologous sequence may be performed using any suitable approach. In one example, the two ends of such a molecule are ligated to each other using a suitable ligase, e.g., a ligase suitable for blunt end ligation or sticky end ligation. Blunt end ligation could be employed by providing a heterologous sequence having a blunt end at one end of the cDNA and a heterologous sequence having a blunt end at the other end of the cDNA. Sticky end ligation could be employed by providing a heterologous sequence having a sticky end at one end of the cDNA and a heterologous sequence having a complementary sticky end at the other end of the cDNA.

In other aspects, the circularized DNA is produced from a cDNA including a first heterologous sequence at a first end and a second heterologous sequence at the end opposite the first end; circularization is achieved using a splint oligonucleotide including sequences complementary to the first and second heterologous sequences; and the known heterologous sequence of the circularized DNA includes the first and second heterologous sequences or portions thereof. In such aspects, a Gibson assembly approach or modified version thereof (e.g., NEBuilder Hifi DNA assembly) could be used to join the ends of the nucleic acid using the splint oligonucleotide.

Once the circularized DNA is produced, the methods further include performing rolling circle amplification using the circularized DNA as template to produce a concatemer comprising repeating segments comprising the cDNA and the known heterologous sequence. As used herein, the term “rolling circle amplification” or “RCA” refers to an amplification (e.g., isothermal amplification) that generates linear concatemerized copies of a circular nucleic acid template using a strand-displacing polymerase. During RCA, the polymerase continuously adds single nucleotides to a primer (e.g., an oligonucleotide primer or a primer produced by nicking a double-stranded circular DNA (e.g., using an endonuclease) annealed to the circular template which results in a concatemer single-stranded (ssDNA) that contains tandem repeats (e.g., tens, hundreds or more tandem repeats) complementary to the circular template. In some embodiments, a primer that hybridizes to the known heterologous sequence or portion thereof is employed. In other aspects, a random primer (e.g., a random hexamer) is employed. Suitable strand-displacing polymerases that may be employed include, but are not limited to, Phi29 polymerase, Bst polymerase, and Vent exo-DNA polymerase. Reagents, protocols and kits for performing RCA are known and include, e.g., the RCA DNA Amplification Kit available from Molecular Cloning Laboratories; and TruePrime RCA Kit available from Expedeon.

The methods further comprise sequencing the concatemer or fragments thereof. In certain embodiments, the concatemer is prepared and sequenced using an approach described in Volden et al. (2018) PNAS 115 (39) 9726-9731 or International Application Publication No. WO 2019/204,720, the disclosures of which are incorporated herein by reference in their entireties for all purposes.

When the methods comprise sequencing fragments of the concatemer, such fragments may be produced using a variety of approaches. In certain embodiments, such fragments are produced by tagmenting the concatemer. As used herein, the term “tagmenting” or “tagmentation” refers to modification of the concatemer by a transposome. By “transposome” is meant a complex of a transposase and a transposon. The transposase may be any suitable transposase, such as a Tn5 transposase, a Tn7 transposase, a Mu transposase, or the like. The transposon may include a sequencing adapter flanked by recognition sequences for the transposase being employed. For example, when the transposome includes a Tn5 transposase, the transposon may include a sequencing adapter flanked by Tn5 transposase recognition sequences (e.g., 19 base pair mosaic end (ME) sequences). Upon contacting the concatemer by a transposome, the transposons of the transposomes are inserted randomly into the concatemer, thereby fragmenting the concatemer. The concatemer is contacted with the transposomes under conditions suitable for insertion of the transposon into the concatemer. Such conditions may vary depending upon the particular transposase employed. Typically, the conditions will include incubating the transposomes and concatemer in a buffered reaction mixture (e.g., a reaction mixture buffered with Tris-acetate, or the like) at a pH of from 7 to 8, such as pH 7.5. The transposome may be provided such that about a molar equivalent, or a molar excess, of the transposon is present relative to the concatemer. Suitable temperatures include from 32 to 42° C., such as 37° C. The reaction is allowed to proceed for a sufficient amount of time, such as from 10 minutes to 3 hours. The reaction may be terminated by adding a solution (e.g., a “stop” solution), which may include an amount of SDS and/or other transposase reaction termination reagent suitable to terminate the reaction. Protocols and materials for achieving fragmentation of nucleic acids using transposomes are available and include, e.g., those provided in the EZ-Tn5™ transpose kits available from Epicentre®, Nextera® kits available from Illumina®, or the like. In some embodiments, the concatemer is tagmented using an approach described in U.S. Patent No. 10,280,449, the disclosure of which is incorporated herein by reference in its entirety for all purposes.

The sequencing may be carried out on any suitable sequencing platform, including a Sanger sequencing platform, a next generation sequencing (NGS) platform (e.g., using a next generation sequencing protocol), or the like. NGS sequencing platforms of interest include, but are not limited to, a sequencing platform provided by Illumina® (e.g., the iSeq 100™, MiniSeq™, MiSeq™, NextSeq 550™, or NextSeq 2000™ sequencing systems); a nanopore-based sequencing platform, non-limiting examples of which include those provided by Oxford Nanopore Technologies (e.g., a MinlON™, GridIONx5™, PromethlON™, or SmidglON™ nanopore-based sequencing system); Ion Torrent™ (e.g., the Ion PGM™ and/or Ion Proton™ sequencing systems); Pacific Biosciences (e.g., the Sequel II sequencing system); Life Technologies™ (e.g., a SOLiD sequencing system); Roche (e.g., the 454 GS FLX+ and/or GS Junior sequencing systems); or any other sequencing platform of interest. Detailed protocols for preparing the tagged extension products and/or fragments thereof for sequencing (e.g., by further amplification (e.g., solid-phase amplification), or the like), sequencing the amplicons, and analyzing the sequencing data are available from the manufacturer of the sequencing system of interest.

In certain embodiments, when the methods of the present disclosure comprise sequencing fragments of the concatemer, the methods comprise tagmenting the concatemer and sequencing the fragments using an Illumina® sequencing system.

According to some embodiments, when the methods of the present disclosure comprise sequencing the concatemer or fragments thereof, the methods comprise sequencing the concatemer or fragments thereof using a nanopore-based sequencing system. In nanopore sequencing, the nanopore serves as a biosensor and provides the sole passage through which an ionic solution on the cis side of the membrane contacts the ionic solution on the trans side. A constant voltage bias (trans side positive) produces an ionic current through the nanopore and drives ssDNA or ssRNA in the cis chamber through the pore to the trans chamber. A processive enzyme (e.g., a helicase, polymerase, nuclease, or the like) may be bound to the polynucleotide such that its step-wise movement controls and ratchets the nucleotides through the small-diameter nanopore, nucleobase by nucleobase. Because the ionic conductivity through the nanopore is sensitive to the presence of the nucleobase’s mass and its associated electrical field, the ionic current levels through the nanopore reveal the sequence of nucleobases in the translocating strand. A patch clamp, a voltage clamp, or the like, may be employed.

Suitable conditions for measuring ionic currents through transmembrane pores (e.g., protein pores, solid state pores, etc.) are known in the art. Typically, a voltage is applied across the membrane and pore. The voltage used may be from +2 V to -2 V, e.g., from -400 mV to +400 mV. The voltage used may be in a range having a lower limit selected from -400 mV, -300 mV, -200 mV, -150 mV, -100 mV, -50 mV, -20mV and 0 mV and an upper limit independently selected from +10 mV, + 20 mV, +50 mV, +100 mV, +150 mV, +200 mV, +300 mV and +400 mV. The voltage may be in the range of from 100 mV to 240mV, e.g., from 120 mV to 220 mV.

The methods are typically carried out in the presence of a suitable charge carrier, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or l-ethyl-3 -methyl imidazolium chloride. Generally, the salt is present in the aqueous solution in the chamber. Potassium chloride (KCI), sodium chloride (NaCI) or cesium chloride (CsCI) may be used, for example. The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M, or from 1 M to 1.4 M. The salt concentration may be from 150 mM to 1 M. The methods are preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of a nucleotide to be identified against the background of normal current fluctuations.

In some embodiments, the rate at which the concatemer or fragments thereof are exposed to the nanopore is controlled using a processive enzyme. Non-limiting examples of processive enzymes that may be employed include polymerases (e.g., a phi29 or other suitable polymerase) and helicases, e.g., a Hel308 helicase, a RecD helicase, a Tral helicase, a Tral subgroup helicase, an XPD helicase, or the like. The concatemer or fragments thereof may be bound by the processive enzyme (e.g., by binding of the processive enzyme to a recognition site present in a sequencing adapter located at an end of the concatemer), followed by the resulting complex being drawn to the nanopore, e.g., by a potential difference applied across the nanopore. In other aspects, the processive enzyme may be located at the nanopore (e.g., attached to or adjacent to the nanopore) such that the processive enzyme binds the concatemer or fragments thereof upon arrival of the concatemer at the nanopore.

The nanopore may be present in a solid-state film, a biological membrane, or the like. In some embodiments, the nanopore is a solid-state nanopore. In other embodiments, the nanopore is a biological nanopore. The biological nanopore may be, e.g., an alpha-hemolysin-based nanopore, a Mycobacterium smegmatis porin A (MspA)-based nanopore, or the like.

Details for obtaining raw sequencing reads of nucleic acid molecules of interest using nanopores are described, e.g., in Feng et al. (2015) Genomics, Proteomics & Bioinformatics 13(1):4-16. Raw sequencing reads may be obtained using, e.g., a MinION™, GridIONx5™, PromethION™, or SmidgION™ nanopore-based sequencing system, available from Oxford Nanopore Technologies. Detailed design considerations and protocols for carrying out nanopore-based sequencing are provided with such systems.

Once a raw sequencing read of the concatemer or fragments thereof is obtained, the present methods further include identifying the repeating segments in the raw sequencing read. In some embodiments, identifying the repeating segments in the raw sequencing read includes identifying at least one sequence of the known heterologous sequence in the raw sequencing read. In certain aspects, the at least one sequence of the known heterologous sequence is identified in the raw sequencing read using a BLAST-Like Alignment Tool (BLAT).

In some embodiments, identifying the repeating segments in the raw sequencing read includes subjecting the raw sequencing read to a modified Smith-Waterman self-to-self alignment. The Smith-Waterman algorithm is a dynamic programming algorithm that performs local sequence alignment for determining similar regions between two strings of nucleic acid or protein sequences. Instead of looking at the entire sequence, the Smith-Waterman algorithm compares segments of all possible lengths and optimizes the similarity measure. In certain aspects, identifying the repeating segments in the raw sequencing read comprises parsing a score matrix of the modified Smith-Waterman self-to-self alignment.

In certain aspects, producing a consensus sequence of the cDNA (e.g., full-length cDNA) includes combining the sequences of the repeating segments using a partial order alignment (POA).

Once a consensus sequence of the full-length cDNA is produced, the methods of the present disclosure may further include subjecting the consensus sequence to error-correction. In some embodiments, subjecting the consensus sequence to error-correction comprises subjecting the consensus sequence to rapid consensus (Racon).

Detailed guidance and example approaches for obtaining a raw sequencing read of a concatemer using a nanopore, identifying repeating segments in the raw sequencing read, producing a consensus sequence of the cDNA based on the sequences of the repeating segments, performing any desired error correction, etc. may be found in the Experimental section below.

The sequences may be produced from immune cell RNAs that encode human leukocyte antigens (HLAs), antibody heavy chains, antibody light chains, a chain of a T cell receptor (e.g., a β chain of a TCR or portion thereof comprising the CDR3 region of the β chain).

According to some embodiments, the cDNA is produced from an immune cell RNA that encodes an HLA, and wherein the method further comprises typing the HLA based on the sequencing. The IPT-IMGT/HLA database contains the systematic names and sequences of thousands of different HLA alleles. The systematic names (e.g. HLA-A*01:01:01:01) contain multiple groups of digits separated by colons to denote the relationship between sequences. The first two digits of an HLA allele name identify the group the allele belongs to (2-digit resolution), the second two digits identify the protein sequence and are affected by non-synonymous variants (4-digit resolution), the third group of digits indicates synonymous variants in the coding region of the protein (6-digit resolution), and the fourth group of digits variants identifies variants outside the protein coding region (8-digit resolution). In some embodiments, typing the HLA based on the sequencing comprises determining HLA A, B, C (MHC class I); DR, DP, DQ types (MHC class II); or a sub-type thereof. Detailed guidance regarding how the methods of the present disclosure may be employed for HLA typing is provided in the Experimental section below.

According to some embodiments, the cDNA is produced from a B cell RNA that encodes a chain of a B cell receptor (e.g., an antibody chain). Such methods may further comprise typing the B cell receptor (e.g., antibody chain) based on the sequencing. By “B cell receptor” or “BCR” is meant immunoglobulin (Ig) expressed by a B-cell. BCRs or Igs are proteins made up of four polypeptide chains, two heavy chains (H chains) from the IGH locus and two light chains (L chains) from either the IGK (kappa) or the IGL (lambda) locus, forming an H₂L₂ structure. Both H and L chains contain a variable domain comprising complementarity determining regions (CDR1-3) involved in antigen recognition, and a constant domain. The H chains of Igs are initially expressed as membrane-bound isoforms using either the IgM or IgD constant region isoform, but after antigen recognition the H chain constant region can class switch to several additional isotypes, including IgG, IgE and IgA. The diversity of naïve Igs within an individual is mainly determined by CDR3. The CDR3 domain of IGH chains is created by the combinatorial joining of the V_{H}, D_{H}, and J_{H} gene segments. CDR domain sequence diversity is further increased by independent addition and deletion of nucleotides at the V_{H}-D_{H}, D_{H}-J_{H}, and V_{H}-J_{H} junctions during the process of IG gene rearrangement. Ig sequence diversity is further augmented by somatic hypermutation (SHM) throughout the rearranged IG gene after a naïve B cell initially recognizes an antigen. The process of SHM is not restricted to CDR3, and therefore can introduce changes in the germline sequence in framework regions, CDR1 and CDR2, as well as in the somatically rearranged CDR3.

According to some embodiments, the cDNA is produced from a T cell RNA that encodes a chain of a T cell receptor, e.g., a β chain of a TCR or portion thereof comprising the CDR3 region of the β chain. Such methods may further include typing the TCR based on the sequencing. By “T cell receptor” or “TCR” is meant a disulfide-linked membrane bound heterodimeric protein normally consisting of the highly variable α and β chains expressed as part of a complex with the invariant CD3 chain molecules. T cells expressing these two chains are referred to as α:β (or αβ) T cells, though a minority of T cells express an alternate receptor, formed by variable γ and σ chains, referred as γσ T cells. TCR development occurs through a lymphocyte specific process of gene recombination, which assembles a final sequence from a large number of potential segments. This genetic recombination of TCR gene segments in somatic T cells occurs during the early stages of development in the thymus. The TCRα gene locus contains variable (V) and joining (J) gene segments (Vα and Jα), whereas the TCRβ locus contains a D gene segment in addition to Vβ and Jβ segments. Accordingly, the α chain is generated from VJ recombination and the β chain is involved in VDJ recombination. This is similar for the development of γδ TCRs, in which the TCRγ chain is involved in VJ recombination and the TCRδ gene is generated from VDJ recombination. The TCR α chain gene locus consists of 46 variable segments, 8 joining segments and the constant region. The TCR β chain gene locus consists of 48 variable segments followed by two diversity segments, 12 joining segments and two constant regions. The D and J segments are located within a relatively short 50 kb region while the variable genes are spread over a large region of 1.5 mega bases (TCRα) or 0.67 mega bases (TCRβ).

Sequence information obtained from the sequencing step of the methods of the present disclosure may be analyzed using one or a variety of approaches, including one or any combination of the sequence analysis approaches described in the Experimental section below.

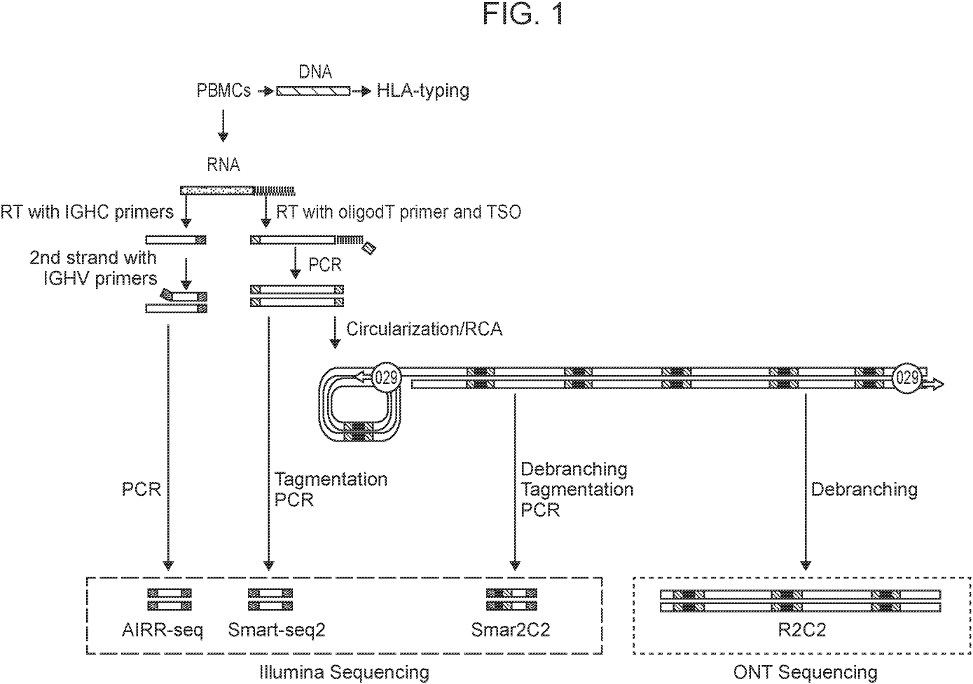

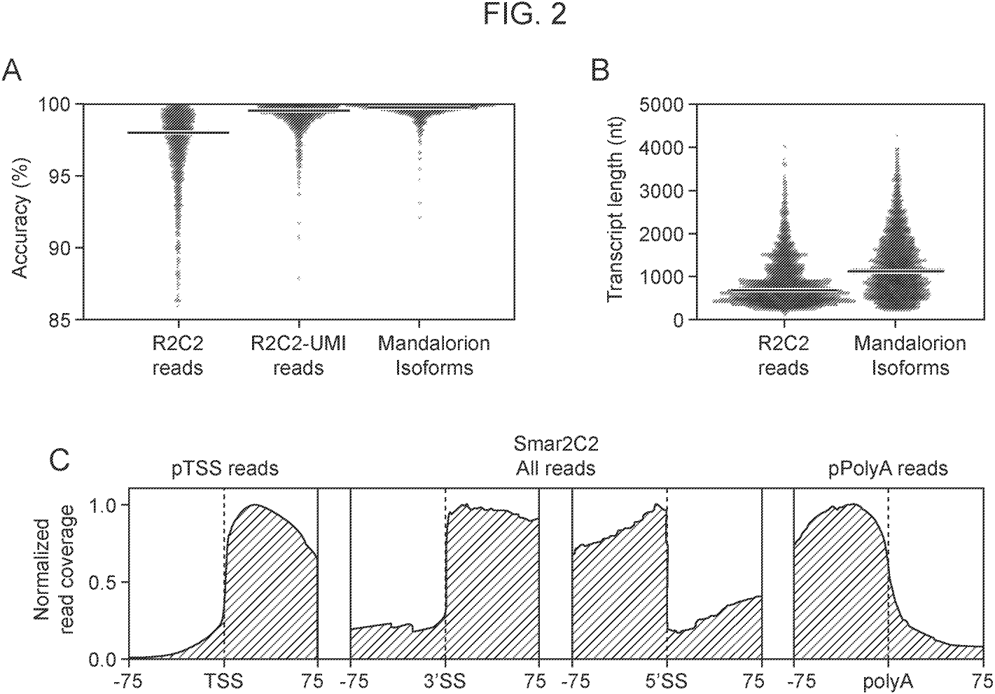

In certain embodiments, the methods of the present disclosure comprise performing two or more of any of the sequencing approaches described herein on an immune cell RNA sample, e.g., two or more of the sequencing approaches schematically illustrated in FIG. 1. According to some embodiments, the methods include performing R2C2 sequencing on an immune cell RNA sample, and further performing one, two, or each of Smar2C2, Smart-seq2, and AIRR-seq on the immune cell RNA sample. In certain embodiments, R2C2 sequencing and Smar2C2 sequencing are performed on the immune cell RNA sample. As noted in the Experimental section below, certain approaches may be advantageous for determining particular sequence and biological information regarding the immune system of the subject from whom the sample is obtained.

### Computer-Readable Media and Systems

Also provided by the present disclosure are computer-readable media and systems. The computer-readable media and systems find use, e.g., in the sequencing and/or analysis of the immune cell RNA sequences obtained from the sequencing. For example, the computer-readable media and systems find use in performing one or any combination of the sequence analysis approaches described in the Experimental section below. As such, one or more steps of the methods may be computer-implemented. By “computer-implemented” is meant the one or more steps is implemented using one or more processors and one or more non-transitory computer-readable media. For example, in certain embodiments, provided are computer-implemented methods for analyzing the immune cell RNA sequences, the methods being implemented using one or more processors and one or more non-transitory computer-readable media comprising instructions stored thereon, which when executed by the one or more processors, cause the one or more processors to analyze the immune cell RNA sequences by one or more sequence analysis approaches described in the Experimental section below.

A variety of processor-based systems may be employed to implement the embodiments of the present disclosure. Such systems may include system architecture wherein the components of the system are in electrical communication with each other using a bus. System architecture can include a processing unit (CPU or processor), as well as a cache, that are variously coupled to the system bus. The bus couples various system components including system memory, (e.g., read only memory (ROM) and random access memory (RAM), to the processor.

System architecture can include a cache of high-speed memory connected directly with, in close proximity to, or integrated as part of the processor. System architecture can copy data from the memory and/or the storage device to the cache for quick access by the processor. In this way, the cache can provide a performance boost that avoids processor delays while waiting for data. These and other modules can control or be configured to control the processor to perform various actions. Other system memory may be available for use as well. Memory can include multiple different types of memory with different performance characteristics. Processor can include any general purpose processor and a hardware module or software module, such as first, second and third modules stored in the storage device, configured to control the processor as well as a special-purpose processor where software instructions are incorporated into the actual processor design. The processor may essentially be a completely self-contained computing system, containing multiple cores or processors, a bus, memory controller, cache, etc. A multi-core processor may be symmetric or asymmetric.

To enable user interaction with the computing system architecture, an input device can represent any number of input mechanisms, such as a microphone for speech, a touch-sensitive screen for gesture or graphical input, keyboard, mouse, motion input, speech and so forth. An output device can also be one or more of a number of output mechanisms. In some instances, multimodal systems can enable a user to provide multiple types of input to communicate with the computing system architecture. A communications interface can generally govern and manage the user input and system output. There is no restriction on operating on any particular hardware arrangement and therefore the basic features here may easily be substituted for improved hardware or firmware arrangements as they are developed.

The storage device is typically a non-volatile memory and can be a hard disk or other types of computer-readable media which can store data that are accessible by a computer, such as magnetic cassettes, flash memory cards, solid state memory devices, digital versatile disks, cartridges, random access memories (RAMs), read only memory (ROM), and hybrids thereof.

The storage device can include software modules for controlling the processor. Other hardware or software modules are contemplated. The storage device can be connected to the system bus. In one aspect, a hardware module that performs a particular function can include the software component stored in a computer-readable medium in connection with the necessary hardware components, such as the processor, bus, output device, and so forth, to carry out various functions of the disclosed technology.

Embodiments within the scope of the present disclosure may also include tangible and/or non-transitory computer-readable storage media or devices for carrying or having computer-executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform tasks or implement abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

### Compositions

Also provided by the present disclosure are compositions. The compositions find use, e.g., in practicing the methods of the present disclosure.

In some embodiments, provided are compositions that comprise one or any combination of reagents useful for producing a circularized DNA comprising an immune cell cDNA (e.g., a splint oligonucleotide), performing rolling circle amplification to produce a concatemer (e.g., a strand-displacing polymerase, an RCA primer, and/or the like), tagmenting the concatemer (e.g., one or more components of a transposome, such as a transposase and/or a transposon), sequencing the concatemer or fragments thereof (primers for adding sequencing adapters, etc.), and/or the like. According to some embodiments, a composition of the present disclosure comprises one or more (e.g., two or more) reagents for performing R2C2 sequencing and/or Smar2C2 on an immune cell RNA sample. Non-limiting examples of such reagents include those described in the Experimental section below.

In certain embodiments, the compositions include the one or more reagents present in a liquid medium. The liquid medium may be an aqueous liquid medium, such as water, a buffered solution, and the like. One or more additives such as a salt (e.g., NaCl, MgCl₂, KCl, MgSO₄), a buffering agent (a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.), a protease inhibitor, glycerol, and the like may be present in such compositions.

Any of the compositions of the present disclosure may be present in a container. Suitable containers include, but are not limited to, tubes, vials, and plates (e.g., a 96- or other-well plate).

### Kits

Also provided by the present disclosure are kits. The kits find use, e.g., in practicing the methods of the present disclosure.

In some embodiments, provided are kits that comprise one or any combination of reagents useful for producing a circularized DNA comprising an immune cell cDNA (e.g., a splint oligonucleotide), performing rolling circle amplification to produce a concatemer (e.g., a strand-displacing polymerase, an RCA primer, and/or the like), tagmenting the concatemer (e.g., one or more components of a transposome, such as a transposase and/or a transposon), sequencing the concatemer or fragments thereof (primers for adding sequencing adapters, etc.), and/or the like. According to some embodiments, a kit of the present disclosure comprises one or more (e.g., two or more) reagents for performing R2C2 sequencing and/or Smar2C2 on an immune cell RNA sample. Non-limiting examples of such reagents include those described in the Experimental section below.

Components of the kits may be present in separate containers, or multiple components may be present in a single container. A suitable container includes a single tube (e.g., vial), one or more wells of a plate (e.g., a 96-well plate, a 384-well plate, etc.), or the like.

The kits may include instructions, such as instructions for performing one or more steps of a method of the present disclosure, e.g., producing a circularized DNA comprising an immune cell cDNA, performing rolling circle amplification to produce a concatemer, tagmenting the concatemer, sequencing the concatemer or fragments thereof, and/or the like. The instructions may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### Experimental

Described herein is a nanopore sequencing-based Rolling Circle to Concatemeric Consensus (R2C2) protocol that enables the generation of over 10,000,000 full-length cDNA sequences at a median accuracy of 97.9%. The dataset was used to demonstrate that deep and accurate full-length cDNA sequencing can be used to provide isoform-level transcriptome analysis for over 9,000 loci. Further, workflows were developed to generate accurate sequences of HLA alleles which - based on the limited number of HLA alleles present in a single individual - indicate that R2C2 based full-length cDNA sequencing could be used for HLA allele typing and discovery. Finally, a strategy to extract detailed AIRR data for the analysis of the adaptive immune system is provided. Importantly, neither HLA nor AIRR analysis require specific knowledge of the diversity at HLA and BCR/TCR loci.

### Example 1 - R2C2 Data Characteristics

Total RNA and genomic DNA were extracted from PBMC samples purified from the whole blood of a healthy male adult. DNA was used for high-resolution HLA-typing whereas total RNA was used for several transcriptome analysis assays. First, full-length cDNA was generated using a modified first half of the Smart-seq2 protocol. cDNA was then split to generate sequencing libraries with three different methods. To generate standard Smart-seq2 libraries, part of this cDNA was tagmented using Tn5. Next, the full-length cDNA was circularized and rolling circle amplification was performed on the resulting circular cDNA. This reaction generated long dsDNA containing multiple concatemeric copies of the original full-length cDNA. We either sequenced this DNA on the ONT MinION® sequencing system using the R2C2 protocol (Volden et al. (2018) PNAS 115 (39) 9726-9731) or tagmented it with Tn5 to generate a hybrid Smart-seq2 and R2C2 short-read library referred to herein as Smar2C2 (FIG. 1). Due to their distinct features the different libraries were intended for different tasks. R2C2 data were intended to provide full-length cDNA sequences for isoform identification and AIRR characterization. Smart-seq2 and Smar2C2 data were intended to provide highly accurate short-read data for SNP identification and isoform sequence polishing, with Smar2C2 data providing improved coverage of transcript ends.

The R2C2 method sequences the same cDNA sequence multiple times to overcome the low accuracy of raw 1D ONT cDNA reads. It does so by circularizing double-stranded cDNA molecules using a DNA splint in a variant of Gibson assembly (NEBuilder, New England Biolabs) and amplifying this circularized cDNA using phi29-based rolling circle amplification (RCA). The resulting RCA product consists of concatemers of the same cDNA sequence that can be sequenced on long-read sequencers and converted into accurate consensus sequences. As disclosed herein, R2C2 can be used in conjunction with unique molecular identifiers (UMIs) as part of the DNA splints. Because the UMIs are linked to cDNA after PCR amplification they do not indicate unique RNA molecules, but instead reflect unique circularization events, thereby allowing combination of raw reads originating from the same cDNA molecule to improve R2C2 consensus read accuracy.

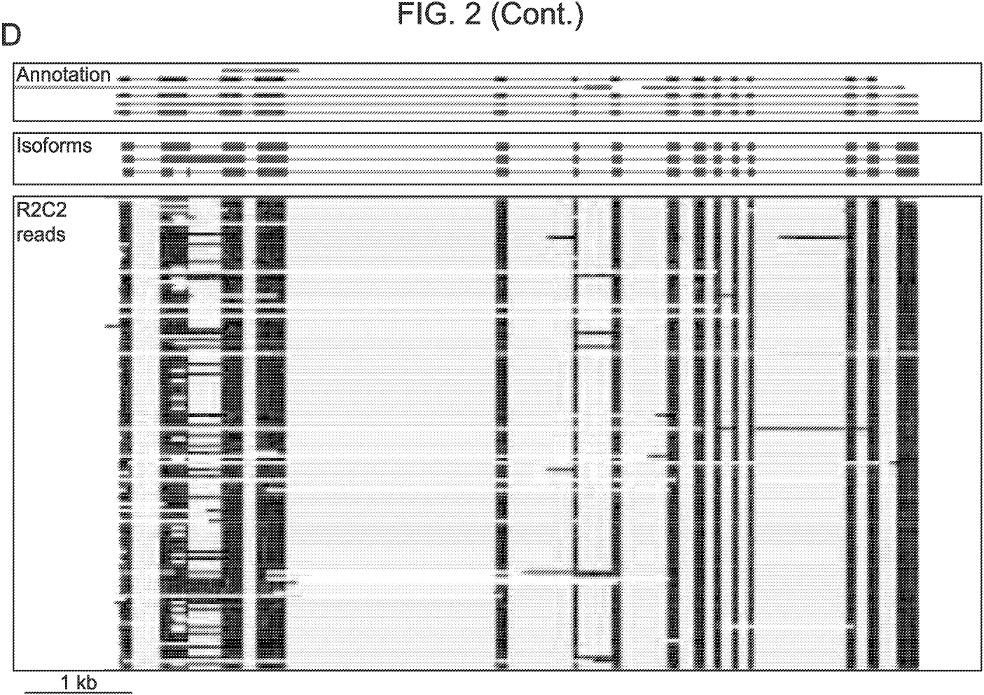

R2C2 data was generated in four technical replicates using individual ONT MinION 9.4.1 flow cells. 1D raw reads from each flow cell were processed using the C3POa program to generate a total of 10,298,086 R2C2 consensus reads (FIG. 2, panel A). Of these reads, 122,353 could be grouped with one or more other reads based on their UMIs and were combined into 58,893 R2C2-UMI reads. Analysis of the resulting reads showed an accuracy of 97.9% for regular R2C2 reads and of 99.3% for R2C2-UMI reads (FIG. 2, panel B). Because of the relatively low number of these R2C2-UMI reads, regular R2C2 reads and R2C2-UMI reads were merged into a single data set with a total number of 10,234,626 reads with a median length of 721 nt (FIG. 2, panel C) of which >99.9% aligned to the human genome.

FeatureCounts was used to quantify gene-level expression based on these R2C2 reads and Smart-seq2 reads. Gene expression as determined by the different protocols showed a Pearson r-value of 0.93 suggesting good correlation between them. While this suggests that R2C2 captures the transcriptome in a quantitative manner it is not the focus of this analysis as the strength of long-read sequencing like R2C2 is not gene-level but instead isoform-level transcriptome analysis.

### Example 2 - Isoform Identification and Evaluation

To identify transcript isoforms, R2C2 reads were input into a revised version of the Mandalorion program (v3, github). A total of 21,358 transcript isoforms expressed from 9,971 gene loci with a median length of 1,152 nt (FIG. 2, panel C) were detected. The isoform sequences Mandalorion produced showed a median accuracy of 99.7%. Their actual accuracy is likely even higher considering the genome sequence of the sample donor is not expected to be identical to the human reference genome sequence.

The quality of these isoforms was further evaluated using SQANTI (FIG. 2, panel A). A total of 12,250 isoforms were scored as ‘full-splice match’ (FSM) of an annotated transcript although with potentially new transcription start and polyA sites. 2,265 isoforms were scored as ‘novel in catalog’ (NIC), meaning that they used annotated splice sites in previously un-annotated combinations. 1,369 isoforms were scored as ‘novel not in catalog’ (NNC), which means that they contain un-annotated splice sites and potentially entirely un-annotated exons. 4,661 isoforms were scored as “incomplete splice match” (ISM) which could mean that they are potential artifacts and were not used to extract potentially new TSS and polyA sites. Alignment starts and ends of FSM, NIC, and NNC isoforms that were more than 10 nt (or 20 nt) away from annotated transcription start sites (TSS) and polyA sites were detected, resulting in 5,525 (20 nt: 3417) new TSSs and 5,712 (20 nt: 5023) new polyA sites. By analyzing the alignments of NNC isoforms 365 new exons that did not overlap with any exon in the GENCODE (v29) annotation were detected. Isoforms that contained new ends (TSS or polyA sites) or exons were based on a median of 21 and 12 R2C2 reads, respectively, while isoforms containing no new features were based on a median of 34 reads. These differences were highly significant with multiple testing corrected MannWhitneyU p-values of 4.57e-95 (Isoforms with new ends vs. isoforms with no new features) and 2.16e-25 (Isoforms with new exons vs. isoforms with no new features). This suggests that isoform features are more likely to be already included in the GENCODE annotation if they are part of highly expressed isoforms.

These new features were then validated using short-read Illumina protocols. Because all general-purpose RNA-seq protocols struggle to capture transcript ends, a new Illumina-based RNA-seq method that overcomes this issue was developed. This method, termed Smar2C2 is a hybrid of the Smart-seq2 and R2C2 methods that tagments not cDNA molecules but the cDNA concatemers generated as part of the R2C2 method (FIG. 1). As a result, Tn5 based tagmentation is not affected by cDNA ends because these ends are now encapsulated within a much larger DNA molecule.

Gene expression as determined by Smar2C2 and Smart-seq2 showed a Pearson r-value of 0.97 suggesting that the Smar2C2 protocol does not distort the cDNA composition. Further, Smar2C2 reads were more likely than Smart-seq2 reads to cover transcript ends in the form of reads containing putative transcription start sites (pTSS reads) or polyA sites (pPolyA reads) (see Methods). Processing of the ~39 million Smart-seq2 and ~23 million Smar2C2 read pairs suggests that Smar2C2 data contains ~6× (Smart-seq2: 3% and Smar2C2 17% of all reads) more pTSS reads and ~25× (Smart-seq2: 0.17% and Smar2C2 4% of all reads) more pPolyA reads than Smart-seq2 data. Smar2C2 read coverage dropped sharply outside new splice sites, and pTSS and pPolyA read coverage also dropped sharply outside new TSS and polyA sites indicating that new isoform features identified by Mandalorion were indeed present within the cDNA pool (FIG. 2, panel D).

The usefulness of deep full-length isoform data was highlighted using the CD19 gene expressed by B cells as an example. CD19 is of great importance because it is a target of cancer immunotherapy in B cell Acute Lymphoblastic Leukemia (ALL) and other B cell cancers. At a total read depth of ~10 million reads, only 146 reads aligned to the CD19 gene. These 146 R2C2 reads appeared to split into 3 major and several minor isoforms (FIG. 2, panel E). Mandalorion identified the 3 major isoforms which confirmed a previously identified splice site in the 2nd exon of CD19 that we recently observed in single B cells. Because they encode distinct proteins, these isoforms may affect whether CAR T-cells can bind leukemia cells expressing them.

### Example 3 - Allele Specific Isoform Expression

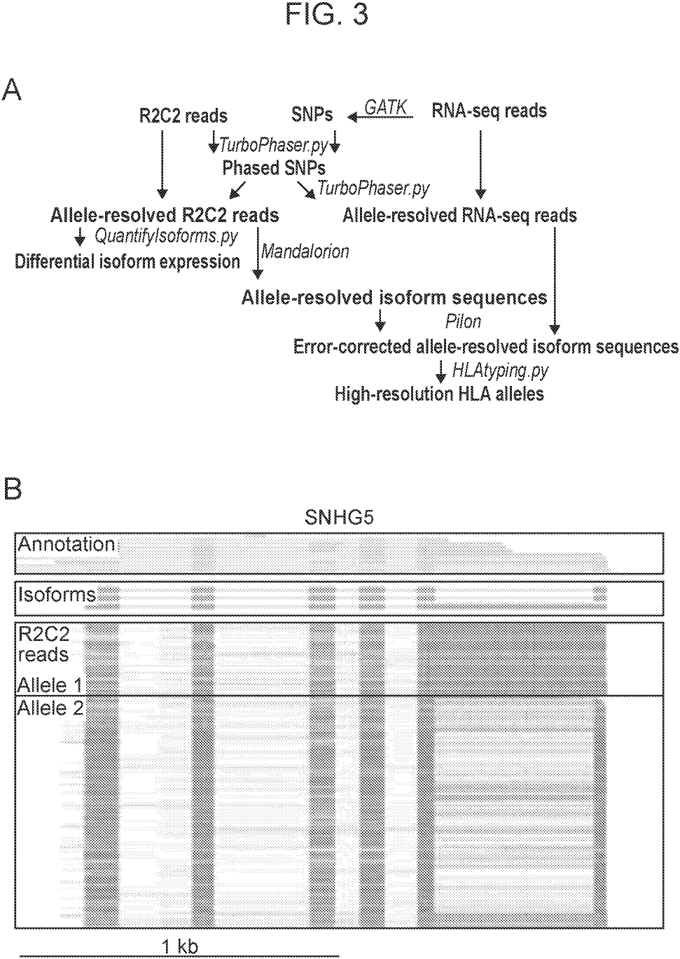

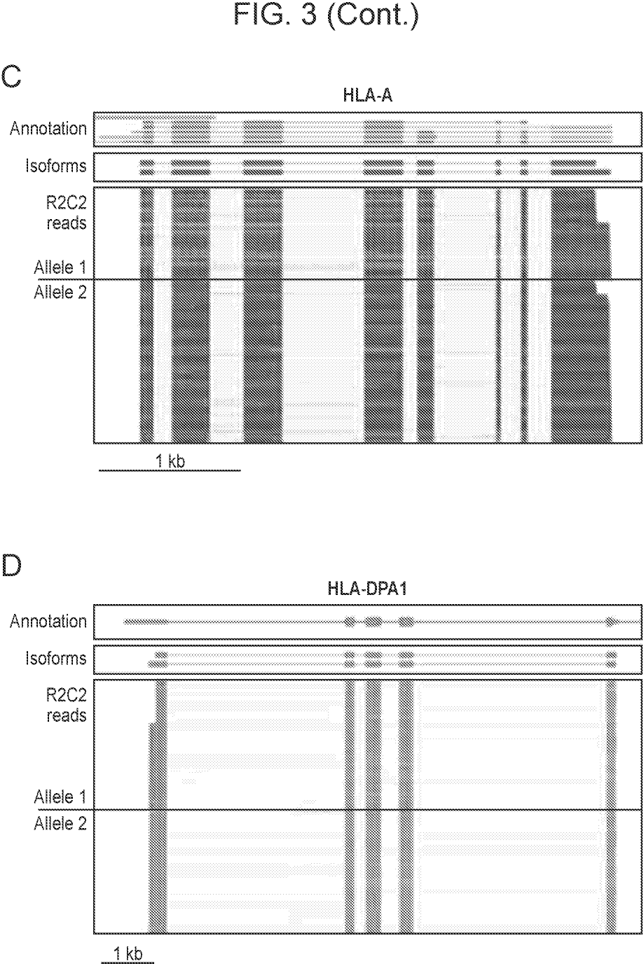

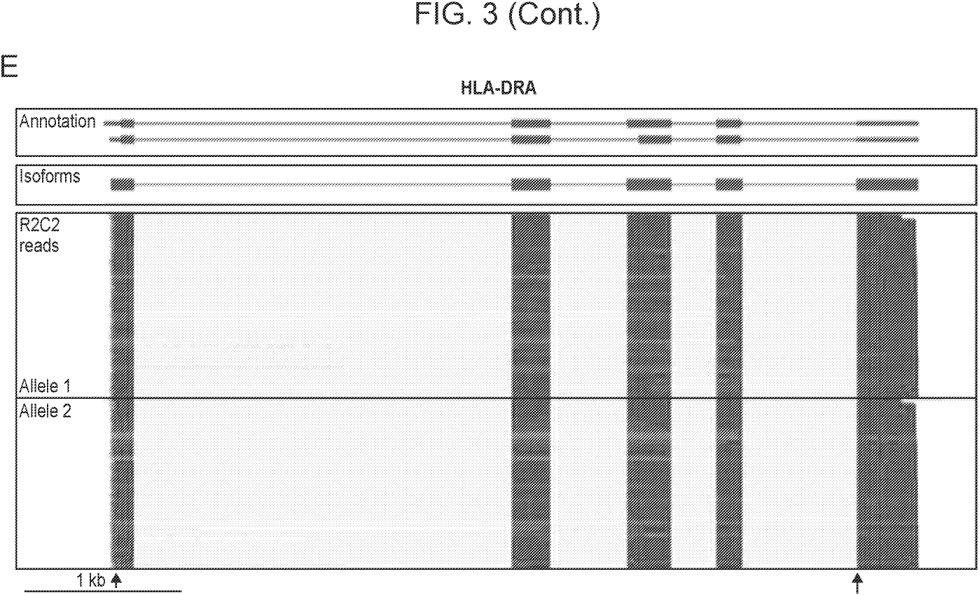

At close to 98% accuracy, R2C2 reads should be well suited for allele-specific isoform expression analysis. Because the SNP identification is much more established with short-read data, the Smart-seq2 and Smar2C2 data produced to identify SNPs present in the PBMC sample were analyzed using the standard Genome Analysis Toolkit (GATK) RNAseq workflow (FIG. 3, panel A). Using the TurboPhaser.py (see Methods) script developed for this purpose, heterozygous SNPs from this list were extracted and these SNPs phased within gene boundaries using R2C2 reads. TurboPhaser.py then sorted R2C2 reads and short-read RNA-seq read pairs into alleles based on the phased SNPs they contained. Overall, 756,072 R2C2 reads (7.4% of all reads - Allele1: 377,794; Allele2: 378,278) and 1,817,151 RNA-seq read pairs (2.9% of all read pairs, Allele1: 872,130; Allele2: 945,021) were assigned to either of two alleles. It is noteworthy that R2C2 reads are more than twice as likely to be sorted into alleles than RNA-seq read pairs based on the same set of phased SNPs. This is likely due to R2C2 reads, in contrast to RNA-seq reads, covering entire transcripts and all SNPs they contain.

Next, the allele-resolved R2C2 reads were used to quantify the expression of the previously identified isoforms. To this end, these reads were separated into four technical replicates based on the flow cells they were generated on. Using DESeq, ~80 isoforms were identified that showed differential expression between the two alleles while accounting for the technical variation associated with each MinIon® run. The SNHG5 gene highlights this differential expression with transcripts of one allele always retaining the first intron of the gene and transcripts of the other allele either splicing or retaining the intron (FIG. 3, panel B). Interestingly, seven of the ~80 differentially expressed isoforms originated from an HLA gene. HLA-A and HLA-DPA1 both show differentially expressed isoforms with alternative polyA sites. While the alternative HLA-A isoform was previously observed, the HLA-DPA1 isoform was not (FIG. 3, panels C and D).

### Example 4 - Allele-Resolved Isoform Sequences Enable High Resolution HLA-Typing

R2C2 reads are suited for identifying which HLA alleles are present in an individual. HLA genes are highly diverse within the human population with different individuals carrying different alleles. Identifying which HLA alleles are present in an individual (HLA-typing) is of utmost importance when establishing donor-recipient compatibility in transplantation medicine. Current RNA-seq based HLA-typing methods rely on databases of previously identified and systematically cataloged HLA alleles. The IPT-IMGT/HLA database contains the systematic names and sequences of thousands of different HLA alleles. The systematic names (e.g. HLA-A*01 :01 :01 :01) contain multiple groups of digits separated by colons to denote the relationship between sequences. The first two digits of an HLA allele name identify the group the allele belongs to (2-digit resolution), the second two digits identify the protein sequence and are affected by non-synonymous variants (4-digit resolution), the third group of digits indicates synonymous variants in the coding region of the protein (6-digit resolution), and the fourth group of digits variants identifies variants outside the protein coding region (8-digit resolution).

Using this database, current RNA-seq based methods can determine the identity of HLA alleles present in an individual with 4 to 6-digit resolution. However, even the most advanced methods like arcasHLA have a 10% error-rate for the identification of some HLA genes and, importantly, cannot determine new HLA alleles absent from the database they use. Reliable HLA typing therefore still requires dedicated DNA-based approaches. These approaches PCR-amplify full-length HLA genes from genomic DNA and determine the sequence of the resulting amplicons. These sequences are then compared to the database of known HLA alleles to determine 6-digit HLA types.

R2C2 should enable a similar approach to targeted DNA-based approaches by determining the full-length sequences of all HLA transcripts present in the analyzed sample without knowledge of what HLA alleles have previously been identified. To test this, the 377,794 R2C2 reads assigned to the first allele and 378,278 R2C2 reads assigned to the second allele were used as separate inputs into Mandalorion which then generated 2,237 and 2056 allele-specific isoforms, respectively. Importantly, Mandalorion generates entirely read-based consensus sequences for each isoform it identifies, which in this case included at least one full-length isoform for each major HLA gene on either allele. Next, to achieve the highest possible accuracy required for identifying variants and achieving unambiguous HLA-typing, allele-resolved RNA-seq reads were used to error-correct the allele-specific Mandalorion isoforms with pilon.

To determine the identity of the HLA alleles present in the analyzed sample, the HLAtyping.py utility of Mandalorion which aligns these error-corrected allele-resolved HLA isoform sequences to the complete database of HLA alleles using minimap2 and extracts the best match for each HLA gene was used. After finding the best HLA allele match of an error-corrected allele-specific isoform, we truncated the match to 6-digit resolution to match clinical high-resolution HLA-typing. All HLA alleles we identified in this way (R2C2+RNA-seq) matched DNA-based high-resolution HLA typing performed at the Immunogenetics and Transplantation Laboratory (ITL) at UCSF (Targeted Amplicon NGS) (Table 1).

The identified HLA alleles were compared to HLA alleles determined using only RNA-seq data and different programs. The seq2HLA package (SS2/seq2HLA) only generates data with 4-digit resolution and failed to identify HLA-DPA1, HLA-DQA1 and HLA-DQB1 as heterozygous and miscalled HLA-DPB1. The arcasHLA package (RNA-seq/arcasHLA) performed better, determining the correct HLA alleles for all HLA genes. Interestingly however, while both RNA-seq/arcasHLA and R2C2+RNA-seq strategies identified only one 6-digit resolution allele for the HLA-DRA genes (HLA-DRA*01:01:01), only R2C2+RNA-seq identified the HLA-DRA gene as heterozygous by identifying distinct sequences for the two alleles. Because Targeted Amplicon NGS data was not available for HLA-DRA to serve as ground truth, it is uncertain whether the R2C2+RNA-seq strategy yields a correct result for this gene. However, visualizing allele-resolved R2C2 reads suggests the existence of heterozygous variants in two distinct HLA-DRA alleles that are several hundred bp apart. ArcasHLA cannot resolve this because the variants are outside the protein coding region, where they affect 8-digit but not 6-digit HLA resolution (FIG. 3, panel E).

Overall, these findings show that accurate full-length cDNA sequencing at high depth allows the determination of highly accurate sequences of HLA alleles which can then be used for high-resolution HLA-typing. In contrast to short-read RNA-seq based HLA-typing which requires a reference database, these HLA allele sequences can be also used to discover so far unknown HLA alleles. Beyond clinical applications, full-length cDNA sequencing therefore represents a potentially valuable research tool for investigating the diversity of the human population which currently requires high coverage whole genome sequencing data.

TABLE 1

R2C2 full-length cDNA sequencing enables high-resolution HLA-typing

| | Allele 1 | | | | Allele 2 | | | |
|---|---|---|---|---|---|---|---|---|
| Data Source | Targeted Amplicon NGS | R2C2+RNA-seq | RNA-seq | RNA-seq | Targeted Amplicon NGS | R2C2+RNA-seq | RNA-seq | RNA-seq |
| Program | HLA Twin | HLAtyping.py | seq2HLA | arcasHLA | HLA Twin | HLAtyping.py | seq2HLA | arcasHLA |
| HLA-A | 03:01:01 | 03:01:01 | 03:01 | 03:01:01 | 32:01:01 | 32:01:01 | 32:01′ | 32:01:01 |
| HLA-B | 35:01:01 | 35:01:01 | 35:01 | 35:01:01 | 39:01:01 | 39:01:01 | 39:01 | 39:01:01 |
| HLA-C | 04:01:01 | 04:01:01 | 04:01 | 04:01:01 | 12:03:01 | 12:03:01 | 12:03 | 12:03:01 |
| HLA-DRA | | 01:01:01 | 01:01 | 01:01:01 | | 01:01:01 | 01:01 | |
| HLA-DRB1 | 16:01:01 | 16:01:01 | 16:01 | 16:01:01 | 01:01:01 | 01:01:01 | 01:01 | 01:01:01 |
| HLA-DPAL | 01:03:01 | 01:03:01 | 02:01 | 01:03:01 | 02:01:01 | 02:01:01 | 02:01 | 02:01:01 |
| HLA-DPB1 | 04:02:01 | 04:02:01 | 105:01 | 04:02:01 | 10:01:01 | 10:01:01 | 10:01 | 10:01:01 |
| HLA-DQA1 | 01:01:01 | 01:01:01 | 01:02 | 01:01:01 | 01:02:02 | 01:02:02 | 01:02 | 01:02:02 |
| HLA-DQB1 | 05:01:01 | 05:01:01 | 05:01 | 05:01:01 | 05:02:01 | 05:02:01 | 05:01 | 05:02:01 |

*HLA alleles were typed using the programs indicated on top. Different programs employed for HLA-typing rely on different data sources. The HLA Twin program requires DNA-based amplicon sequencing (Targeted Amplicon NGS), Our HLA-typing.py program requires isoform sequences generated using full-length cDNA (R2C2) and polished with RNA-seq sequences. The seq2HLA and arcasHLA program require only RNA-seq sequences. DRA was not evaluated by Targeted Amplicon NGS. Contradicting results are shown in italics.

### Example 5 - Extracting Adaptive Immune Receptor Repertoires (AIRR) From R2C2 Data

Evaluated next was whether accurate full-length sequencing could also - completely or in part - replace specialized assays for the analysis of adaptive immune receptor repertoires. To do so, R2C2 reads were extracted from a data set that aligned to BCR or TCR gene loci. Because each read could be derived from a unique AIRR transcript, every read had to be treated independently and isoform determination was not reliable. Further, since the loci present in the human genome sequence are not the rearranged loci found in each individual B or T cell, these alignments were not evaluated directly. Instead the sequences of these reads were annotated using the IgBlast algorithm which identifies V, (D), and J segments, CDR3 sequences at the V(D)J intersection, and mutations present in each sequence. Finally, sequence similarity was used to determine which constant region is present in each sequence. In this way, tens of thousands of adaptive immune receptor sequences were identified (Table 2 - number of reads that aligned to the respective locus and could be annotated as AIRR transcripts using IgBlast is shown).

TABLE 2

Full-length adaptive immune receptor repertoires can be extracted from R2C2 whole transcriptome data

| Receptor | Gene | Number of R2C2 reads |
|---|---|---|
| BCR | IGH | 12,863 |
| | IGL | 26,759 |
| | IGK | 24,460 |
| TCR | TRA | 7,289 |
| | TRB | 13,118 |
| | TRD | 316 |
| | TRG | 551 |

In-depth analysis was performed on these annotations with a focus on BCR heavy chain (IgH) sequences which are the only adaptive immune receptor undergoing VDJ recombination, somatic hypermutation, and class-switch recombination. This makes BCR IgH sequences the biggest analysis challenge and therefore the best and most conservative target for detailed validation.

The R2C2-based BCR IgH repertoires were compared to 266,390 BCR IGH sequences generated from the same RNA sample by the gold-standard UMI-based targeted AIRR-seq method disclosed herein (called AIRR-seq herein). A total of 3,261 BCR IGH sequences generated from a different RNA sample from the same individual that was published previously was also used. These sequences were generated using a previously described TMI-seq method which, in contrast to standard AIRR-seq, succeeds in covering the entire V segment by overcoming Illumina read length limitations through a combination of Tn5-based tagmentation and unique molecular identifiers. The analysis was focused on the most relevant features of the BCR repertoire, namely 1) CDR3 length, 2) Isotype usage, 3) V segment composition and usage, 4) Somatic Hypermutation, and 5) Clonality.

### CDR3 Length

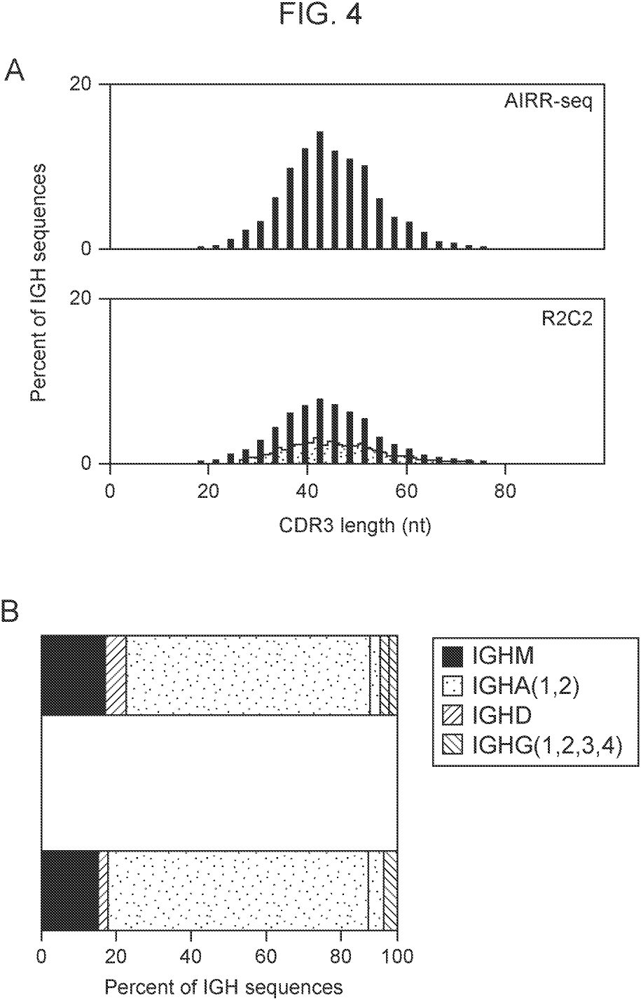

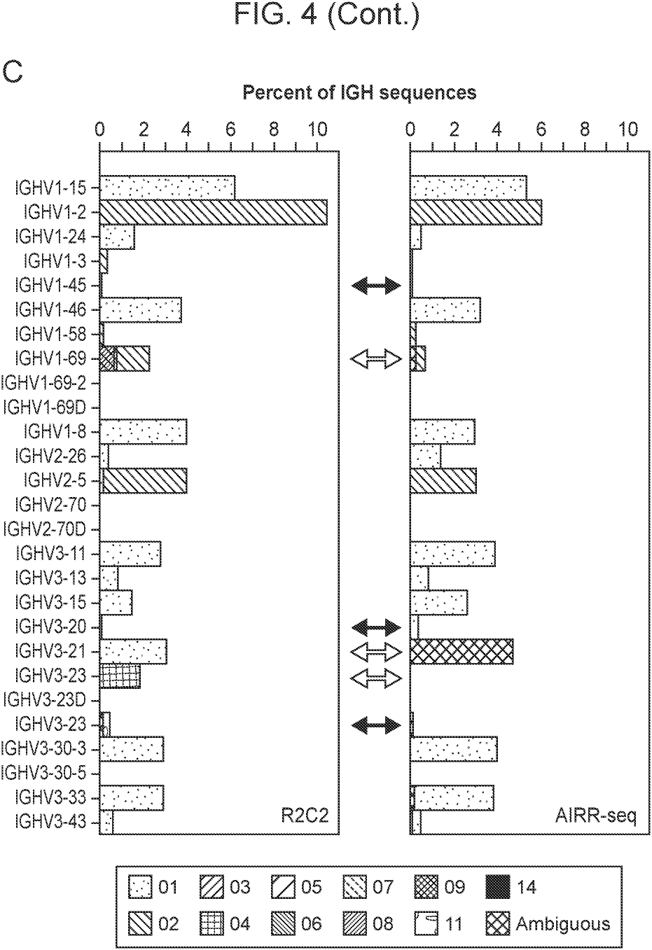

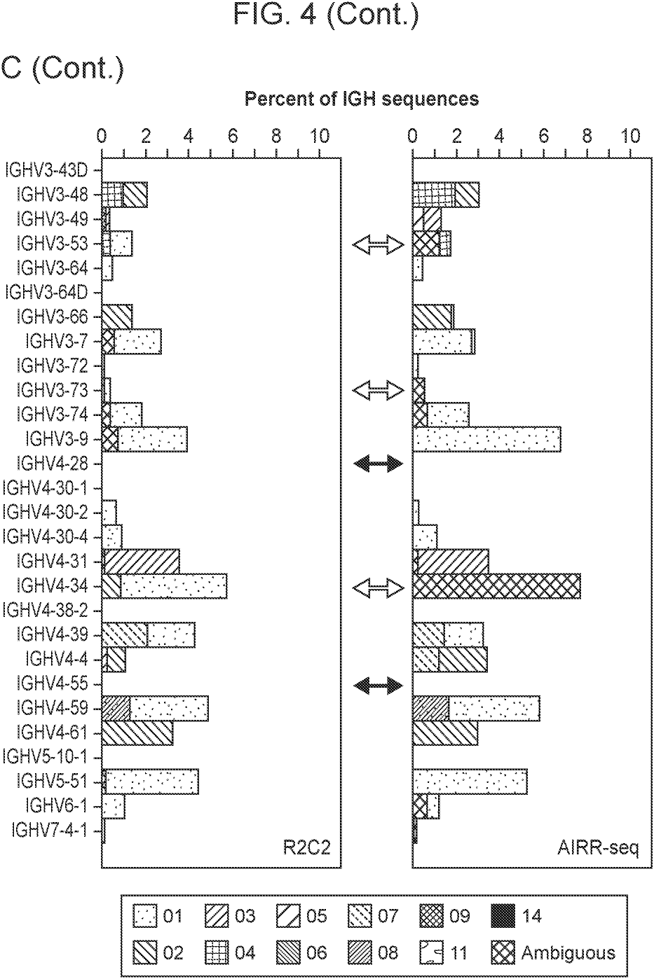

R2C2-based BCR IGH repertoires capture the full width of CDR3 lengths. The sequences of CDR3s are responsible for the majority of B-cell receptor/antibody specificity and are composed of semi-random sequences at the intersection of V, D, and J segments. However, CDR3 sequences in functional antibodies are limited to certain lengths to maintain the reading frame between variable and constant regions. This limitation can be clearly observed in CDR3 lengths of AIRR-seq sequences but is less pronounced for R2C2 sequences. This is likely due to remaining Indel errors in the CDR3 sequences (FIG. 4, panel A). So, although R2C2-based repertoires capture CDR3 length of the appropriate mean length and distribution, downstream analyses that rely on CDR3 length to differentiate functional and non-functional BCR sequence would be hampered.

### Isotype Usage

R2C2-based BCR IGH repertoires can be used to determine B cell isotype usage. Isotype usage reflects the activity of the adaptive immune system at a given time. IgM and IgD sequences are mostly expressed by naive B cells, while IgA, IgG and IgE sequences are only expressed by previously activated B cells that have undergone class-switch recombination. Relative isotype expression has been shown to change upon vaccine administration, infection, or immunosuppression.

Isotype usage was similar between AIRR-seq and R2C2-derived repertoires suggesting that R2C2-derived repertoires will be able to determine the immune activation state of an individual faithfully (FIG. 4, panel B). Improving upon any AIRR sequencing approach currently available, R2C2-derived repertoires also resolve whether a BCR IgH transcript encodes for a membrane-bound or secreted (antibody) protein. This is possible because R2C2 reads cover the entire 3′ end of IgH transcripts where this alternative splicing event occurs. Isoform-level information showed that the majority of IgM transcript are membrane-bound (Membrane (M): 1261; Secreted (S): 417) while, surprisingly, IgD transcripts were split evenly between the two isoforms (M: 123; S:164). As expected, over 95% of the sequences of IgA and IgG isotype subtypes were secreted (e.g. IgA1: M:182; S:8113).

### V Segment Composition and Usage

R2C2 repertoires can be used to investigate V segment usage. The adaptive immune receptor genes loci (BCR IgH, Igλ, and Igκ and TCR TRA, TRB, TRD, and TRG) are among the most diverse and complex in the human genome. Each BCR IgH locus is thought to contain 40-50 V segments and individuals diverge in which V segments and V segment alleles they possess. AIRR data can determine which V segments are present in an individual’s genome and how often they are recombined. However, standard AIRR-seq assays most often use PCR primers within V segments, thereby masking variation underneath the priming site and missing variation beyond it. TMI-seq addressed this by priming in the Leader exon beyond the V segment exon, however it still relied on potentially biased multiplex primers and requires complex molecular biology and computational workflows.

Because the R2C2-based repertoires could not be successfully analyzed with computational tools meant for virtually error-free AIRR-seq, we determined V segment composition in the analyzed individual by simply counting how many reads were scored by IgBlast as using a specific V segment with three or fewer mismatches. Reads that assigned equally well to different alleles of the same V segment were counted as ambiguous while reads aligned equally well to different V segments were discarded for this analysis. A V segment allele was counted as detected in a repertoire if it was seen in at least two sequences and accounted for at least 20% of sequences of the V segment.

In general, AIRR-seq and R2C2 showed similar recombination frequencies for V segments. However, it was found that the deeper AIRR-seq data has an advantage when detecting V segment alleles for V segments that are rarely recombined, including IGHV1-45 (0.08% of IGH sequences in AIRR-seq data), IGHV3-20 (0.3%), IGHV3-30 (0.04%), IGHV4-28 (0.01%), IGHV4-55 (0.02%) which R2C2 did not detect at our requisite abundance of three independent reads. However, we found that R2C2 can unambiguously detect V segments that AIRR-seq could not (FIG. 4, panel C). AIRR-seq could not detect one or two of the alleles for IGHV1-69, IGHV3-21, IGHV3-23, IGHV3-53, and IGHV3-73 while R2C2 could. Importantly, all alleles detected by R2C2 were also detected by TMI-seq data.

This analysis shows that, although specialized AIRR-seq protocols have an edge when detecting V segments that are rarely recombined, it produces incomplete and therefore often ambiguous V segment sequences. Although the TMI-seq method we developed can produce full-length V segment data, it requires a complex workflow and is therefore unlikely to be employed for routine clinical analysis. Overall, R2C2 presents an appealing set of tradeoffs and is therefore a promising tool for determining the V segment composition and usage within a sample.

### Somatic Hypermutation

R2C2 reads are accurate enough to detect mutations in IgH sequences introduced by somatic hypermutation. This was demonstrated by comparing mutations in BCR transcript sequences which can undergo somatic hypermutation with TCR transcript sequences which are expected to be entirely free of somatic mutations.

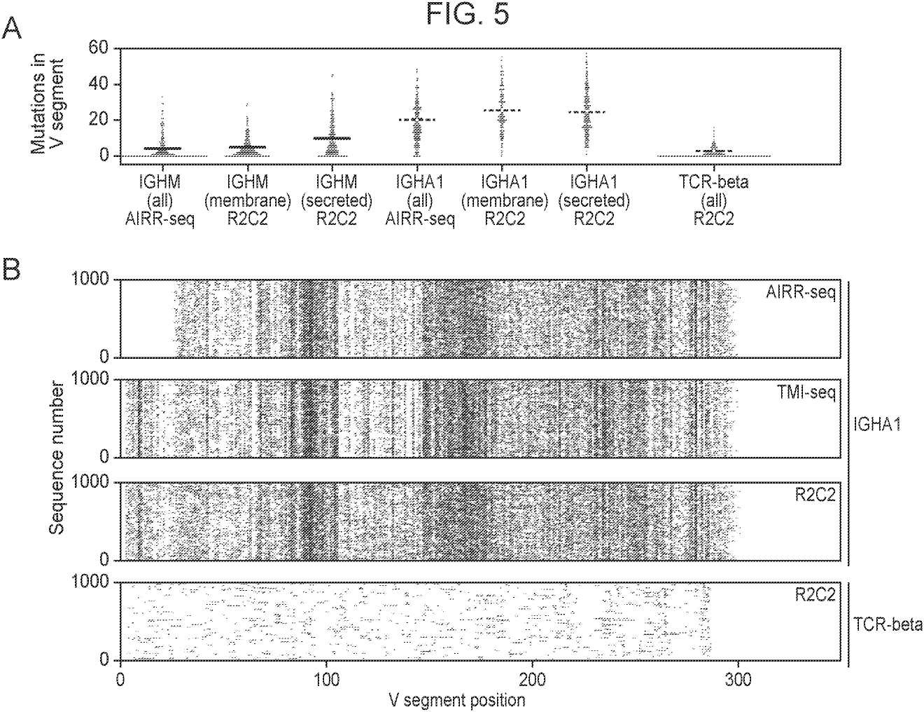

The analysis focused on mismatches which are by far the most common result of somatic hypermutation. The R2C2 reads did show only about two mismatches per 300nt TCR which corresponds to a mismatch-rate of 0.6% and is in line with the remaining 2% total error-rate in R2C2 reads being mostly composed of indels. Two mismatches per V segment can therefore be seen as background error in the potential mutated BCR sequences that were next analyzed. IgM transcripts are thought to be mostly expressed by naive unmutated B cells but can be also undergo somatic hypermutation. R2C2 reads can distinguish membrane-bound and secreted (antibodies) isoforms of BCR transcripts. Secreted IgM sequences contained more mutations than membrane-bound IgM sequences, indicating that they are more likely to be expressed by B cells that have undergone activation and somatic hypermutation (FIG. 5, panel A). This difference in mismatch levels disappears in IgA1 BCR sequences which are known to be expressed only by B cells that have undergone activation and somatic hypermutation.

Mismatch levels were higher in R2C2-derived IgA1 BCR sequences than in AIRR-seq derived IGHA1 BCR sequences (Average 24.89 to 20.19, Monte-Carlo Permutation test p-value<0.00001). Adding randomly sampled R2C2-specific background-level mismatches observed in TCR-beta sequences as well as mismatches observed in the first 20 bases of R2C2 IGHA1 BCR sequences to AIRR-seq sequences does not abolish this significant difference (Average 24.89 to 23.8, Monte-Carlo Permutation test p-value<0.00001). One possible explanation of this may be that the primers employed by AIRR-seq fail at binding highly mutated V segments which then are not amplified and detected. In turn, this would indicate that R2C2 might have an advantage when investigating highly mutated sequences like those involved in the immune response to HIV.

Finally, like AIRR-seq and TMI-seq, IGHA1 transcript sequenced by R2C2 show the mutational pattern characteristic of somatic hypermutation with mutational hotspots in CDR1 and CDR2. In contrast to AIRR-seq which uses amplicons primed from FR1 in the BCR transcript, TMI-seq and R2C2 sequence detect mutations all the way to the beginning of the V segment (FIG. 5, panel B).

### Clonality

R2C2-based repertoires can be used to capture the clonal composition of B cells in a sample. The BCR IgH sequences in a sample can be organized into clones (or lineages), i.e. sequences that are expressed by B cells belonging to the same B cell clone. B cell clones originate from a single naive B cell that is activated and starts proliferating. This proliferation is most often associated with somatic hypermutation and class-switching. Big lineages are therefore likely to be composed of class-switched sequences with similar but not identical mutation patterns. Most importantly, they have highly similar CDR3 sequences which can be used to group IgH sequences into lineages computationally. This analysis was performed using AIRR-seq and R2C2-based repertoires.

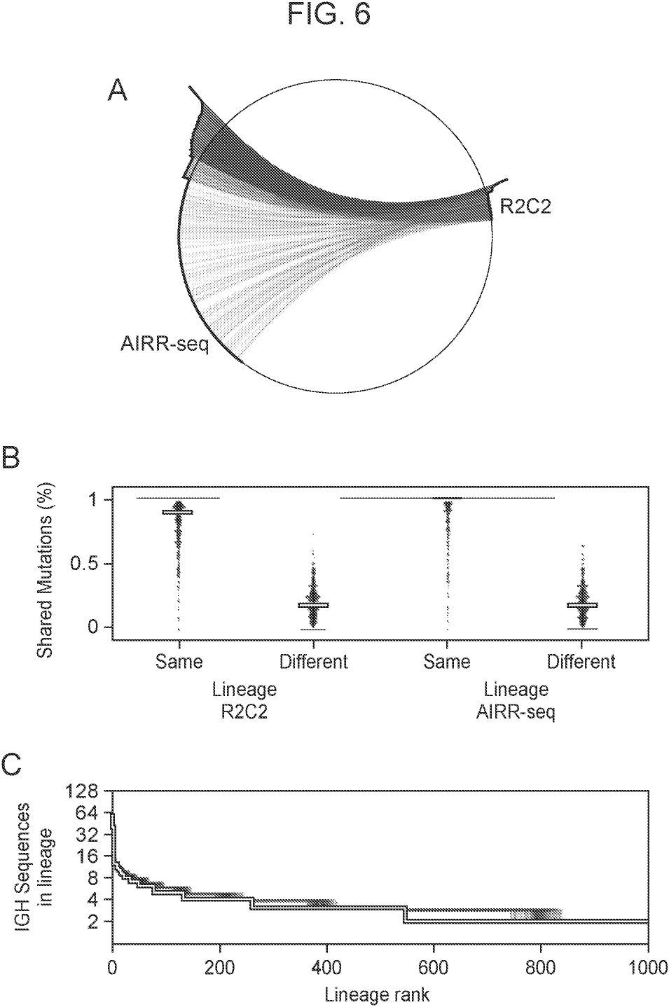

The lineages in the two repertoires were closely related (FIG. 6, panel A). 178 of the top 200 lineages in the AIRR-seq repertoire were also present in the R2C2-based repertoire with missing lineages likely being explained by the lower depth of the R2C2-based repertoire. As expected, most of these repertoires had class-switched to the IgA1 isotype and many contained additional lineage-specific mutations.

Next, these mutations were used to confirm that IgH sequences were not spuriously grouped into lineages. To this end, the percentage of mutations in each IgH sequence shared with other sequences within the same lineage or sequences in different sequences was determined. For both R2C2 and AIRR-seq sequences, this percentage was much higher when comparing sequences within the same lineage confirming the overall accuracy of the disclosed lineage grouping approach (FIG. 6, panel B). Finally, to see whether AIRR-seq and R2C2-based repertoires behave similarly when grouped into lineages, the AIRR-seq repertoire was repeatedly subsampled to the depth of the R2C2-based repertoire before grouping the subsampled sequences into lineages (FIG. 6, panel C). The resulting AIRR-seq lineages were slightly larger than R2C2-based lineages at the same depth indicating that the 2% residual sequencing error present in R2C2 reads causes some related sequences to not be grouped into lineages.

Overall, analysis of AIRR-seq and R2C2-based lineages shows high concordance between the two methods. Of clinical relevance, R2C2-based lineages should be more than capable of tracking B cell clones within and between samples to, for example, track minimal residual disease in leukemia.

In its entirety, the analysis of the ability of R2C2 to investigate BCR IgH repertoires shows that R2C2 can replace specialized AIRR-seq for many purposes. It can be used to determine V segment compositions, isotype usage, and somatic hypermutation levels and patterns, as well as the clonal structure of a population of B cells. Moreover, accuracy of primary nanopore sequencing data is improving at a tremendous pace suggesting that Indel and other errors will rapidly decrease. Finally, while thisR2C2/AIRR-seq comparison was focused on BCR IgH as the most complex and challenging to analyze AIR repertoire, the disclosed results suggest that R2C2-based repertoires for the other adaptive immune receptor loci are likely to be of comparable quality.

### Methods

### Sample Collection and Preparation

All experiments were approved by the Internal Review Board at the University of California Santa Cruz. Two whole blood samples were collected from a healthy human adult volunteer by the University of California Santa Cruz Student Health Center approximately six months apart. Samples were processed by Ficoll gradient (GE Healthcare) to extract PBMCs which were stored in liquid Nitrogen. RNA was extracted from one PBMC sample using the RNeasy mini kit (Qiagen). DNA was extracted from the other PBMC sample using the MagAttract HMW DNA kit (Qiagen).

### HLA-Typing

HLA-typing was performed at the Immunogenetics and Transplantation Laboratory at University of California, San Francisco.

### Sample Preparation

DNA was quantified with NanoDrop (ThermoFisher Scientific, CA, USA) and adjusted to a concentration of 30 ng/µL. Quality of DNA was assessed by measuring absorbance at A₂₃₀, A₂₆₀ and A₂₈₀. DNA samples were amplified by long-range PCR using the Omixon Holotype HLA genotyping kit, generating full-length gene amplicons for HLA-A, B, C, DRB1, DRB3, DRB4, DRB5, DQA1, DQB1, DPA1 and DPB1 loci. Following PCR, amplicons were cleaned with Exo-SAP (Affymetrix, Santa Clara, CA, USA), quantified with QuantiFluor dsDNA system (Promega, Madison, WI, USA), and normalized to approximately 70 ng/µL.

### Library Preparation and Sequencing

Sequencing libraries were generated for each sample using the Omixon Holotype HLA Genotyping Kit (Omixon, Inc. Budapest, Hungary). In brief, libraries from individual HLA amplicons were prepared by enzymatic fragmentation, end repair, adenylation, and ligation of indexed adaptors. The indexed libraries were pooled and concentrated with Ampure XP beads (Beckman Coulter) prior to fragment size selection using a PippinPrep™ (Sage Science, Beverly, MA, USA), selecting a range of fragments between 650 and 1300 bp. The size-selected library pool was quantified by quantitative PCR (qPCR; Kapa Biosystems, Wilmington, MA, USA) and adjusted to 2 nmol/L. The library was then denatured with NaOH and diluted to a final concentration of 8 pmol/L for optimal cluster density and 600 µL was loaded into the MiSeq reagent cartridge (v2 500 cycle kit). The reagent cartridge and flow cell were placed on the Illumina MiSeq (Illumina, San Diego, CA, USA) for cluster generation and 2 × 250 bp paired-end sequencing. Samples were demultiplexed on the instrument and the resulting FASTQ files were used for further analysis. HLA genotyping was assigned using TwinTM version 2.0.1 (Omixon, Inc. Budapest, Hungary) and IMGT/HLA database version 3.24.0_2, using 16000 read-pairs.

### Transcriptome Sequencing Library Preparation

Approximately 200 ng of total RNA was used to generate full-length cDNA using a modified Smart-seq2 protocol. In short, RNA was reverse transcribed using Smartscribe RT (Clontech) and ISPCR-OligodT primer ISPCR-TSO (Table S1). Remaining RNA and primer dimers were digested and cDNA was PCR amplified using RNAseA, Lambda Exonuclease (NEB) and Kapa Biosystems HiFi HotStart ReadyMix (2X) (KAPA) with the following heat-cycling protocol: 37° C. for 30 minutes, 95° C. for 30 seconds followed by 12 cycles of (98° C. 20 seconds; 67° C. 15 seconds; 72° C. for 10 minutes). The reaction was then purified using SPRI beads at a 0.85:1 ratio and eluted in H-₂O. The resulting full-length cDNA was then used as input into Smart-seq2, R2C2, and Smar2C2 library preparation protocols.

### Smart-Seq2

Full-length cDNA was then tagmented with Tn5 enzyme custom loaded with Tn5ME-A/R and Tn5ME-B/R adapters. The Tn5 reaction was performed using 50 ng of cDNA in 5ul, 1 µl of the loaded Tn5 enzyme, 10 µl of H₂O and 4 µl of 5× TAPS-PEG buffer and incubated at 55° C. for 5 min. The Tn5 reaction was then inactivated by the addition of 5 µl of 0.2% sodium dodecyl sulphate and 5 µl of the product was then nick-translated at 72° C. for 6 min and further amplified using KAPA Hifi Polymerase (KAPA) using Nextera_Primer_A and Nextera_Primer_B (Table S1) with an incubation of 98° C. for 30 s, followed by 13 cycles of (98° C. for 10 s, 63° C. for 30 s, 72° C. for 2 min) with a final extension at 72° C. for 5 min. The resulting Illumina library was size selected on an agarose gel to be within 200-400bp and sequenced on a Illumina Nextseq 2×150 run.

### R2c2

For splint generation, 23 ul of H2O, 25 ul of Kapa Biosystems HiFi HotStart ReadyMix (2X) (KAPA), 1 ul of UMI_Splint_Forward (100 uM) and 1 ul of UMI_Splint_Reverse (100 uM) were incubated at 95° C. for 3 minutes, 98° C. for 1 minute, 62° C. for 1 minute, and 72° C. for 6 minutes). The DNA splint was then purified with the Select-A-Size DNA Clean and Concentrator kit (Zymo) with 85 ul of 100% EtOH in 500 ul of DNA binding buffer.

For circularization of cDNA, 200 ng of cDNA was mixed with 200 ng of DNA splint and 2× NEBuilder HiFi DNA Assembly Master Mix (NEB) was added at the appropriate volume. This mix was incubated at 50 C for 60 minutes. To this reaction we added 5ul of NEBuffer 2, 3 ul Exonuclease I, 3 ul of Exonuclease III, and 3 ul of Lambda Exonuclease (all NEB) and adjusted the volume to 50 ul using H₂O. This reaction was then incubated 37° C. for 16 hr followed by a heat inactivation step at 80° C. for 20 minutes. Circularized DNA was then extracted using SPRI beads with a size cutoff to eliminate DNA <500 bp (0.85 beads:1 sample) and eluted in 40 µL of ultrapure H₂O.

For rolling circle amplification, circularized DNA was split into four aliquots of 10 µL, and each aliquot was amplified in its own 50-µL reaction containing Phi29 polymerase (NEB) and exonuclease resistant random hexamers (Thermo) [5 µL of 10× Phi29 Buffer, 2.5 µL of 10 uM (each) dNTPs, 2.5 µL random hexamers (10 uM), 10 µL of DNA, 29 µL ultrapure water, 1 µL of Phi29]. Reactions were incubated at 30° C. overnight. T7 Endonuclease was added to each reaction which were then incubated at 37° C. for 2 h with occasional agitation. The debranched DNA was then extracted using SPRI beads at a 0.5 :1 ratio and eluted in 50 µL of H2O.

For nanopore sequencing, the resulting DNA was sequenced across four separate ONT MinION 9.4.1 flow cells. For each run, 1ug of DNA was prepared using the LSK-109 kit according to the manufacturer’s instructions with only minor modifications. End-repair and A-tailing steps were both extend from 5 minutes to 30 minutes. The final ligation step was also extended to 30 minutes. Each run took 48 hours and the resulting data in Fast5 format was basecalled using the high accuracy model of the gpu accelerated Guppy algorithm (version 2.3.5+53a111f, config file: dna_r9.4.1_450bps_flipflop.cfg). To generate R2C2 consensus reads, the resulting raw reads were processed using the C3POa pipeline (https://github.com/rvolden/C3POa).

### Smar2C2

Library prep for this protocol is highly similar to Smart-seq2, however instead of cDNA, it uses the debranched rolling circle amplified DNA that is composed of cDNA concatemers. 50 ng of this DNA was tagmented with Tn5 enzyme custom loaded with Tn5ME-A/R and Tn5ME-B/R adapters. The Tn5 reaction was performed using 50 ng of cDNA in 5 uL, 1 µL of the loaded Tn5 enzyme, 10 µl of H₂O and 4 µl of 5× TAPS-PEG buffer and incubated at 55° C. for 5 min. The Tn5 reaction was then inactivated by the addition of 5 µl of 0.2% sodium dodecyl sulphate and 5 µl of the product was then nick-translated at 72° C. for 6 min and further amplified using KAPA Hifi Polymerase (KAPA) using Nextera_Primer_A and Nextera_Primer_B (Table S1) with an incubation of 98° C. for 30 s, followed by 13 cycles of (98° C. for 10 s, 63° C. for 30 s, 72° C. for 2 min) with a final extension at 72° C. for 5 min. The resulting Illumina library was size selected on an agarose gel to be within 200-400bp and sequenced on an Illumina Nextseq 2×150 run.

### AIRR-Seq

200 ng of total RNA was used for cDNA Smartscribe (Clontech) first-strand synthesis using a primer pool specific to the first exon of all IGH isotypes (IGHM, IGHD, IGHG1-4, IgGHA1-2, IGHE; see Table S1). In a two-cycle PCR reaction, second and third cDNA strands were synthesized using Kapa Biosystems HiFi HotStart ReadyMix (2X) and two modified primer pools complementary to the beginning of the Framing region 1 (FR1) of the V segment and ~100 bp into the first exon of all IgH isotypes. All primers used in this two-cycle PCR reaction were modified to have unique molecular identifiers and partial Nextera sequences on their 5′ end. cDNA was purified and size-selected to >300 nt using Select-A-Size DNA Clean and Concentrator kits (Zymo). In a 20-cycle PCR reaction, the cDNA is then amplified with primer completing Nextera sequences as well as Illumina i5 and i7 indexes to enable multiplexing of the libraries. Libraries were then sequenced on the Illumina MiSeq using a 2×300 run.

### Gene Expression

R2C2 reads were aligned to the hg38 version of the human genome using minimap2 using “-ax splice” and “--secondary=no” flags and other standard settings. Smart-seq2 and Smar2C2 reads were aligned to the same genome sequence using STAR (version 2.7.1a) and an index built using the gencode v29 annotation gtf file. Read alignments were converted in gene expression counts using featureCounts.

### Identifying Smar2C2 Reads That Contain Transcript Ends

To identify Smar2C2 reads containing putative transcription start sites (pTSS reads) we determined whether a read contained the sequence of template switch oligo which, absent premature template switching, should denote a transcription start site.

To identify Smar2C2 reads containing putative polyA sites (pPolyA reads) read pairs were analyzed. First, it was determined whether one read of a pair contained the sequence of the oligodT primer including a stretch of Ts making the other read of that pair a candidate. We then determine whether a candidate read contained a stretch of Ts which, absent mispriming of the oligodT primer, should denote a polyA site.

### Isoform Analysis

R2C2 reads were analyzed to identify isoforms using version 3 of Mandalorion and standard settings. Isoforms were categorized using the sqanti_qc.py script of the SQANTI program with slight modifications to make it compatible with Python3 and using the gencode (v29) annotation of the human genome. Isoform features were extracted from the categorized isoforms using the ProcessSqantiClassification.py (Mandalorion utility) script as follows. To identify new transcript features, we relied on SQANTI classification:

1) New gene loci were defined by grouping overlapping isoforms that were classified as ‘intergenic’.

2) New TSSs and polyA-sites were defined by identifying alignment starts and ends of isoforms that were classified as ‘full-splice_match’, ‘novel_in_cataiog’, ‘and novel_not_in_catalog’. New TSSs and polyA-sites had to be located more than a chosen distance cutoff away from an annotated TSS or polyA-site.

3) New exons were defined by analyzing isoforms classified as ‘novel_not_in_catalog’. New exons had to have no overlap with annotated exons, i.e. partial or extended known exons were not considered.

### Allele-Specific Isoforms

SNP present in the sample donor’s genome were identified using RNA-seq (Smart-seq2+Smar2C2) read alignments and GATK (version 3.8-1-0-gf15c1c3ef) following the standard RNA-seq SNP identification workflow (https://software.broadinstitute.org/gatk/documentation/article.php?id=3891). Homozygous SNPs were discarded. The remaining heterozygous SNP were phased using R2C2 reads and the new Mandalorion utility TurboPhaser.py, taking advantage of R2C2 reads spanning entire gene loci and grouping SNPs that appeared in the same reads. TurboPhaser.py also sorted R2C2 reads and RNA-seq reads into alleles based on the SNPs they contained. The sorted R2C2 reads were then used in the Mandalorion pipeline to identify isoform sequences. The sequences were then error-corrected using pilon and RNA-seq reads aligned to the isoform sequences using minimap2 with the “-x sr” preset.

### HLA-Typing

Using the HLAtyping.py script Allele-specific error-corrected isoform sequences were aligned to the human genome using minimap2 (“--secondary=no -x splice”). Isoforms aligning to HLA loci were then realigned to HLA transcript sequences retrieved from the IPD-IMGT/HLA database using minimap2 (“-ax splice -N 100”). For each HLA gene and allele, the best full-length match was reported. For RNA-seq-only approaches, Smart-seq2 reads and Smar2C2 reads were pooled and processed as required by seq2HLA and arcasHLA and the programs were run with standard settings.

### AIRR Analysis

R2C2 reads aligning to adaptive immune receptor loci were extracted using samtools. The sequences were then analyzed using IgBlast with output format 19 with V, D, and J segments retrieved from IMGT. For the in-depth analysis of BCR IGH repertoires this output was then parsed using custom scripts to report CDR3 length and Vsegment, as well as the positions of mismatches in the V segments. Isotype, and isoform (secreted vs membrane-bound) of each sequence were determined by matching the part of extracted R2C2 reads matching the constant region was compared to a database of isotype and isoform sequences and, if it was high quality enough, the best match was used. Sequences were then grouped into lineages using a simple single linkage clustering approach using a 90% CDR3 nucleotide similarity cut-off.

### Data Access

All raw sequencing data generated in this study have been submitted to the SRA under accession number PRJNA559668 (R2C2 and RNAseq data) and PRJNA291102 (TMI-seq data). Processed data is available at users.soe.ucsc.edu/~vollmers/PBMC_data/R2C2_reads.fa and users.soe.ucsc.edu/~vollmers/PBMC_data/AIRRseq_reads.fasta

### Code Access

Mandalorion and its utilities for isoform identification and sequence determination are available on github (https://github.com/rvolden/Mandalorion-Episode-III) and in the supplement. The Mandalorion package also contains scripts for processing SQANTI classification, sorting R2C2 reads into alleles, and HLA-typing.

C3POa has been published and described previously. C3POa generates R2C2 consensus reads from ONT raw reads and is available on github (https://github.com/rvolden/C3POa). The C3POa github also contains scripts for identifying and merging reads with matching UMIs. Scripts for the parsing and grouping AIRR data into lineages is available at (https://github.com/christopher-vollmers/AIRR).

### Data Visualization

All data visualization was done using Python/Numpy/Scipy/Matplotlib. Schematics were drawn in Inkscape.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

1. An immune cell ribonucleic acid (RNA) sequencing method, comprising:
producing a circularized DNA comprising a complementary DNA (cDNA) and a known heterologous sequence, wherein the cDNA is produced from an immune cell RNA;
performing rolling circle amplification using the circularized DNA as template to produce a concatemer comprising repeating segments comprising the cDNA and the known heterologous sequence; and
sequencing the concatemer or fragments thereof.

2. The method of claim 1, wherein the cDNA is produced from an immune cell RNA that encodes a human leukocyte antigen (HLA), an antibody heavy chain, an antibody light chain, or a chain of a T cell receptor.

3. The method of claim 1, wherein the sequencing is by single molecule sequencing.

4. The method of claim 1, wherein the sequencing comprises sequencing fragments of the concatemer.

5. The method of claim 4, further comprising, subsequent to performing rolling circle amplification, tagmenting the concatemer to produce the fragments.

6. The method of claim 1, wherein the cDNA is produced from an immune cell RNA that encodes an HLA, and wherein the method further comprises typing the HLA based on the sequencing.

7. The method of claim 1, wherein the cDNA is produced from an immune cell RNA that encodes an antibody chain, and wherein the method further comprises typing the antibody chain based on the sequencing.

8. The method of claim 1, wherein the cDNA is produced from an immune cell RNA that encodes a chain of a T cell receptor, and wherein the method further comprises typing the chain of the T cell receptor based on the sequencing.

9. A method, comprising:
producing immune cell RNA sequencing reads using a Rolling Circle Amplification to Concatemeric Consensus (R2C2) sequencing method;
extracting HLA reads from the sequencing reads; and
producing allele-specific HLA sequences from the extracted HLA reads.

10. The method of claim 9, further comprising comparing the allele-specific HLA sequences to the sequences of known HLA alleles.
